SEQUENCE LISTING (15 sequences)

SEQ ID NO: 1 - protein, 233 aa, Artificial sequence
  hrRNASET2 (full length)
MDKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 2 - protein, 184 aa, Artificial sequence
  hrtrRNASET2-50
EGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 3 - protein, 164 aa, Artificial sequence
  hrtrRNASET2-70
MRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 4 - DNA, 555 bp, Artificial sequence
  Human trT2-50 RNASET2 mutant (optimized for E. coli sequence)
gaaggctgtaatcgtagctggccgttcaacctggaagaaatcaaagatctgttaccggaaatgcgcgcgtattggccggatgtgattcacagctttccgaaccgtagccgcttttggaaacacgaatgggaaaaacatggcacctgtgccgcccaggttgatgccctgaacagccagaaaaaatatttcggccgctctctggaactgtatcgtgaactggatctgaacagcgttctgctgaaactgggcattaaaccgagcatcaactattatcaggtggcggatttcaaagatgccctggcgcgcgtttatggtgtgattccgaaaattcagtgtctgccgccgagccaggatgaagaagttcagaccatcggccagattgaactgtgtctgaccaaacaggatcagcaactgcagaattgtaccgaaccgggtgaacagccgagcccgaaacaggaagtttggctggcgaacggcgccgccgaaagccgtggtctgcgtgtttgtgaagatggcccggttttttatccgccgccgaaaaaaaccaaacactaa

SEQ ID NO: 5 - DNA, 495 bp, Artificial sequence
  Human trT2-70 RNASET2 mutant (optimized for E. coli sequence)
atgcgcgcgtattggccggatgtgattcacagctttccgaaccgtagccgcttttggaaacacgaatgggaaaaacatggcacctgtgccgcccaggttgatgccctgaacagccagaaaaaatatttcggccgctctctggaactgtatcgtgaactggatctgaacagcgttctgctgaaactgggcattaaaccgagcatcaactattatcaggtggcggatttcaaagatgccctggcgcgcgtttatggtgtgattccgaaaattcagtgtctgccgccgagccaggatgaagaagttcagaccatcggccagattgaactgtgtctgaccaaacaggatcagcaactgcagaattgtaccgaaccgggtgaacagccgagcccgaaacaggaagtttggctggcgaacggcgccgccgaaagccgtggtctgcgtgtttgtgaagatggcccggttttttatccgccgccgaaaaaaaccaaacactaa

SEQ ID NO: 6 - DNA, 5518 bp, Artificial sequence
  pHIS-Parallel3
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgtcgtactaccatcaccatcaccatcacgattacgatatcccaacgaccgaaaacctgtattttcagggcgccatggggatccggaattcaaaggcctacgtcgacgagctcactagtcgcggccgctttcgaatctagagcctgcagtctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 7 - DNA, 42 bp, Artificial sequence
  Single strand DNA oligonucleotide
ccatgggtcacgatgataaaatgcgcgcgtattggccggatg

SEQ ID NO: 8 - DNA, 24 bp, Artificial sequence
  Single strand DNA oligonucleotide
gcggccgcaagcttggatccttag

SEQ ID NO: 9 - DNA, 48 bp, Artificial sequence
  Single strand DNA oligonucleotide
ccatggggtgacgatgataaagaaggctgtaatcgtagctggccgttc

SEQ ID NO: 10 - DNA, 24 bp, Artificial sequence
  Single strand DNA oligonucleotide
gcggccgcaagcttggatccttag

SEQ ID NO: 11 - DNA, 702 bp, Artificial sequence
  Recombinant Human RNASET2 optimized for E. coli sequence
atggataaacgtctgcgtgataaccacgaatggaaaaaactgatcatggttcagcattggccggaaaccgtgtgcgaaaaaatccagaacgattgccgtgatccgccagattattggaccattcatggcctgtggccggataaaagcgaaggctgtaatcgtagctggccgttcaacctggaagaaatcaaagatctgttaccggaaatgcgcgcgtattggccggatgtgattcacagctttccgaaccgtagccgcttttggaaacacgaatgggaaaaacatggcacctgtgccgcccaggttgatgccctgaacagccagaaaaaatatttcggccgctctctggaactgtatcgtgaactggatctgaacagcgttctgctgaaactgggcattaaaccgagcatcaactattatcaggtggcggatttcaaagatgccctggcgcgcgtttatggtgtgattccgaaaattcagtgtctgccgccgagccaggatgaagaagttcagaccatcggccagattgaactgtgtctgaccaaacaggatcagcaactgcagaattgtaccgaaccgggtgaacagccgagcccgaaacaggaagtttggctggcgaacggcgccgccgaaagccgtggtctgcgtgtttgtgaagatggcccggttttttatccgccgccgaaaaaaaccaaacactaa

SEQ ID NO: 12 - DNA, 595 bp, Artificial sequence
  Recombinant DNA comprising trT2-50 added with an EK recognition site to be cloned into pHis-parallel3
ccatgggtgacgatgataaagaaggctgtaatcgtagctggccgttcaacctggaagaaatcaaagatctgttaccggaaatgcgcgcgtattggccggatgtgattcacagctttccgaaccgtagccgcttttggaaacacgaatgggaaaaacatggcacctgtgccgcccaggttgatgccctgaacagccagaaaaaatatttcggccgctctctggaactgtatcgtgaactggatctgaacagcgttctgctgaaactgggcattaaaccgagcatcaactattatcaggtggcggatttcaaagatgccctggcgcgcgtttatggtgtgattccgaaaattcagtgtctgccgccgagccaggatgaagaagttcagaccatcggccagattgaactgtgtctgaccaaacaggatcagcaactgcagaattgtaccgaaccgggtgaacagccgagcccgaaacaggaagtttggctggcgaacggcgccgccgaaagccgtggtctgcgtgtttgtgaagatggcccggttttttatccgccgccgaaaaaaaccaaacactaaggatccaagcttgcggccgc

SEQ ID NO: 13 - DNA, 535 bp, Artificial sequence
  Recombinant DNA comprising trT2-70 added with an EK recognition site to be cloned into pHis-parallel3
ccatgggtgacgatgataaaatgcgcgcgtattggccggatgtgattcacagctttccgaaccgtagccgcttttggaaacacgaatgggaaaaacatggcacctgtgccgcccaggttgatgccctgaacagccagaaaaaatatttcggccgctctctggaactgtatcgtgaactggatctgaacagcgttctgctgaaactgggcattaaaccgagcatcaactattatcaggtggcggatttcaaagatgccctggcgcgcgtttatggtgtgattccgaaaattcagtgtctgccgccgagccaggatgaagaagttcagaccatcggccagattgaactgtgtctgaccaaacaggatcagcaactgcagaattgtaccgaaccgggtgaacagccgagcccgaaacaggaagtttggctggcgaacggcgccgccgaaagccgtggtctgcgtgtttgtgaagatggcccggttttttatccgccgccgaaaaaaaccaaacactaaggatccaagcttgcggccgc

SEQ ID NO: 14 - protein, 215 aa, Artificial sequence
  Deduced amino acid sequence of hrtrRNASET2-50 -HIS-EK translation product
MSYYHHHHHHDYDIPTTENLYFQGAMGDDDKEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 15 - protein, 195 aa, Artificial sequence
  Deduced amino acid sequence of hrtrRNASET2-70 -HIS-EK translation product
MSYYHHHHHHDYDIPTTENLYFQGAMGDDDKMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

(19) United States
(12) Patent Application Publication
(10) Pub. No.: US 2011/0207667 A1
(43) Pub. Date: Aug. 25, 2011

(54) EFFICIENT EXPRESSION OF TRUNCATED HUMAN RNASET2 IN E. COLI

(75) Inventors: Oded Shoseyou, Karmei Yoseef (IL); Betty Schwartz, Rechovot (IL); Levava Roiz, Kiryat-Ono (IL); Patricia Smirnoff, Rehovot (IL); Liron Nuttman, Rechovot (IL)

(73) Assignee: Yissum Research Development Company of the Hebrew University of Jerusalem Ltd.

(21) Appl. No.: 13/126,494

(22) PCT Filed: Oct. 29, 2009
(86) PCT No.: PCT/IL09/01012

§ 371 Date: Apr. 28, 2011

Related U.S. Application Data
(60) Provisional application No. 61/193,134, filed on Oct. 30, 2008.

(51) Int. Cl. A61K 38/17; C07K 14/435; C07H 21/04; C12N 1/21; C07K 16/18; A61P 35/00

(52) U.S. Cl. 514/13.3; 530/350; 536/23.5; 435/252.33; 530/387.9

(57) ABSTRACT

An isolated, recombinant truncated human RNASET2 having anti-angiogenic properties, methods for efficient expression thereof in bacteria and therapeutic uses thereof.

### FIELD AND BACKGROUND OF THE INVENTION

The invention described herein relates to truncated human RNASET2, methods for efficient expression thereof in bacteria and the use thereof, specifically, to the actin-binding and anti-tumor and anti-angiogenic properties of the truncated human RNASET2.

Human RNASET2 is a T₂-RNase glycoprotein encoded by the RNASET2 gene which is located on chromosome 6 (6q27) and known as a tumor repressor gene (Trubia et al. 1997. Genomics 42:342-344; Acquati et al. 2001. Meth Mol Biol 160:87-101). Mutation and loss of function of RNASET2 has been associated with increased tumorogenicity and cancer, including carcinomas of the ovary, breast, uterus, stomach, liver,colon/rectum, kidney and hematologic malignancies, such as non-Hodgkin, B-cell lymphoma and acute lymphoblastic leukemia (Smirnoff et al, Cancer 2006, 107:2760-69). Expression of RNASE6PL cDNA in tumor cell lines suppressed tumorogenicity and metastatic potential of cancer cells injected into a suitable host.

The RNASET2 gene was previously cloned into the yeast Pichia pastoris, producing a human recombinant RNASET2. The human recombinant RNASET2 (hrRNASET2) proved to be effective in inhibiting the development of tumor and angiogenic blood vessel in animal models (Smirnoff et al. 2006. Cancer, 107(12), 2760-2769).

The tumor suppressive and anti-angiogenic effect of human RNASET2 is not mediated by its ribonuclease activity. Acquati et al. (Int. J. Onc. 2005; 26:1159-68) demonstrated that a double point mutation (H65/118F) replacing histidine with a phenylalanine resulted in significant loss of ribonucleolytic activity but did not affect RNASET2-mediated suppression of tumorigenesis and metastasis. Smirnoff et al (Cancer, 2006; 107:2760-2769) autoclaved P. pastoris-expressed human recombinant RNASET2, effectively inactivating the ribonucleolytic activity of the enzyme, but without diminishing the actin binding and anti-angiogenic properties.

U.S. Pat. No. 6,590,075 by Human Genome Sciences (Ruben et al) discloses the isolation and cloning of nucleotide sequences for 70 human genes of secreted proteins, including a gene having homology to human RNASET2 (identified as “Gene 47”).

US Patent Application 20090074830 to Hunter et al. discloses the use of a number of anti-angiogenic microtubule-disrupting agents, such as Paclitaxel, encapsulated and prepared in microspheres, for the treatment of a wide variety of angiogenesis-related diseases.

PCT WO 2006/035439 to Roiz et al discloses the cloning and expression, in P. pastoris, of human RNASET2, having tumor suppressing and anti-angiogenic activity in-vivo and in-vitro, as well as having strong actin-binding properties. None of these properties were associated with the ribonucleolytic properties of the protein.

### SUMMARY OF THE INVENTION

According to an aspect of some embodiments of the invention there is provided an isolated human truncated RNASET2 devoid of ribonucleolytic activity and having an anti-angiogenic activity.

According to another aspect of some embodiments of the invention, there is provided a purified preparation of a human truncated RNASET2 devoid of ribonucleolytic activity and having an anti-angiogenic activity.

According to yet another aspect of some embodiments of the invention there is provided a pharmaceutical composition comprising a human truncated RNASET2 devoid of ribonucleolytic activity and having an anti-angiogenic activity, and a pharmaceutically acceptable carrier.

According to still another aspect of some embodiments of the invention there is provided an isolated polynucleotide encoding a truncated human RNASET2 devoid of ribonucleolytic activity and having an anti-angiogenic activity.

According to some embodiments of the invention, the isolated polynucleotide comprises the nucleic acid sequence as set forth in SEQ ID NOs: 4, 5, 12 or 13.

According to one aspect of some embodiments of the invention there is provided an expressible nucleic acid construct comprising an isolated polynucleotide encoding a truncated human RNASET2 devoid of ribonucleolytic activity and having an anti-angiogenic activity.

According to some embodiments of the invention, expression of the nucleic acid construct in bacteria produces at least 50 mg human truncated RNASET2 per liter bacterial culture.

According to some aspects of some embodiments of the invention there is provided a cell transformed with an expressible nucleic acid construct comprising an isolated polynucleotide encoding a truncated human RNASET2 devoid of ribonucleolytic activity and having an anti-angiogenic activity.

According to some embodiments the cell is an E. coli bacterial cell.

According to some aspects of some embodiments of the invention there is provided a bacterial culture comprising a plurality of cells transformed with an expressible nucleic acid construct comprising an isolated polynucleotide encoding a truncated human RNASET2 devoid of ribonucleolytic activity and having an anti-angiogenic activity and expressing at least 50 mg truncated human RNASET2 per liter culture

According to some aspects of some embodiments of the invention there is provided use of a truncated human RNASET2 devoid of ribonucleolytic activity and having an anti-angiogenic activity for inhibiting angiogenesis in a subject in need thereof.

According to some aspects of some embodiments of the invention there is provided use of the truncated human RNASET2 devoid of ribonucleolytic activity and having an anti-angiogenic activity for the manufacture of a medicament for inhibiting angiogenesis in a subject in need thereof.

According to some embodiments, inhibiting angiogenesis is inhibiting angiogenesis of a tumor. The tumor can be a benign or malignant tumor. The tumor can be a primary tumor. The tumor can be a metastatic tumor.

According to yet another aspect of some embodiments of the invention, there is provided an antibody recognizing a human truncated RNASET2 polypeptide having an amino acid sequence as set forth in SEQ ID NOs: 2, 3, 14 or 15.

According to some embodiments, the human truncated T2 RNase is devoid of the amino acid sequence corresponding to amino acid residues 1-32 of the N-terminus of SEQ ID NO: 1.

According to some embodiments, the human truncated RNASET2 comprises an amino acid sequence at least 95% identical to, or as set forth in SEQ ID NO: 2.

According to some embodiments, the human truncated RNASET2 is devoid of the amino acid sequence corresponding to amino acid residues 1-49 of the N-terminus of SEQ ID NO: 1.

According to some embodiments, the human truncated RNASET2 is devoid of the amino acid sequence corresponding to amino acid residues 1-52 of the N-terminus of SEQ ID NO: 1.

According to some embodiments, the human truncated RNASET2 comprises an amino acid sequence at least 95% identical to, or as set forth in SEQ ID NO: 3.

According to some embodiments, the human truncated RNASET2 is devoid of the amino acid sequence corresponding to amino acid residues 1-69 of the N-terminus of SEQ ID NO: 1.

According to some embodiments, the human truncated RNASET2 is devoid of a cysteine residue at least one of amino acid coordinates corresponding to amino acid residue 25, 32 or 52 of the N-terminus of SEQ ID NO: 1.

According to some embodiments, the human truncated RNASET2 is devoid of cysteine residues at amino acid coordinates corresponding to amino acid residues 25 and 32 of the N-terminus of SEQ ID NO: 1.

According to some embodiments, the human truncated RNASET2 is devoid of cysteine residues at amino acid coordinates corresponding to amino acid residues 25, 32 and 52 of the N-terminus of SEQ ID NO: 1.

According to some embodiments, the human recombinant truncated RNASET2 further comprises a recognition entity peptide sequence. The recognition entity peptide sequence can be a His tag.

According to some embodiments, the human truncated RNASET2 has actin binding activity.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the complete protein sequence of RNASET2 (SEQ ID NO: 1). Glu50 and Met70 residues, which constitute the starting points of the truncated forms human recombinant truncated RNASE (hrtrRNASE) T2-50 (SEQ ID NO: 2) and hrtrRNASET2-70 (SEQ ID NO: 3), respectively, are underlined with a single line. The RNase catalytic sites are underlined with a double line. The cysteine residues (in grey) are linked by disulfide bonds;

FIGS. 2A and 2B show a diagrammatic representation of the cloning and expression of truncated human RNASET2 in E. coli. The truncated cDNA sequences encoding for hrtrRNASET2-50 (cys6 573 bp, SEQ ID NO:4) and hrtrRNASET2-70 (cys5 513 bp, SEQ ID NO:5) were prepared optimized for expression in E. coli, cloned into the vector pHis3Parallel (FIG. 2B, SEQ ID NO:6) and expressed in E. coli;

FIG. 3 is a photograph of the SDS-PAGE analysis of both truncated forms of human recombinant RNASET2 and its insert-less pHis3parallel vector (Mock), produced as inclusion bodies in E. coli. Each lane represents 10 μl of crude cell lysate. Lane 1—Molecular markers; Lane 2—Mock; Lane 3—hrtrRNASET2-50 (SEQ ID NO: 14); Lane 4—hrtrRNASET2-70 (SEQ ID NO: 15). Note the heavy bands in lanes 3 and 4;

FIG. 4 is a photograph of the SDS-PAGE analysis of purified hrtrRNASE T2 protein following immobilized metal (HisTrap™) affinity column using AKTA-FPLC™ separation system. Each lane represents 10 μl of the eluted fractions. The recombinant proteins were eluted with an imidazol gradient in equilibration buffer. Lane 1—Molecular markers; Lane 2—Mock; Lane 3—hrtrRNASET2-50 (SEQ ID NO: 14); Lane 4—hrtrRNASET2-70 (SEQ ID NO: 15);

FIGS. 5A and 5B show the actin binding capability of hrtrRNASE T2. FIG. 5A is a photograph showing the SDS-PAGE separation of hrtrRNASE T2 following actin binding in solution. Actin (10 μg) was mixed with 10 μg hrtrRNASE T2-50 (SEQ ID NO: 14) in 20 μl Buffer G. After crosslinking with EDC, each mixture (18 μl) was analyzed by SDS-PAGE and stained with Coomassie Blue to visualize the proteins or 1 μl sample mixture was loaded and exposed to rabbit anti-hrtrRNASE T2-50 or rabbit anti-actin for immunodetection of hrtrRNASE T2-50 or actin, respectively. The arrow indicates the 63-kDa binding complex formation. Lane 1, molecular markers; Lanes 2,3,4: Coomassie blue staining for proteins; Lanes 5,6,7: Immunostaining using anti-hrtrRNASET2-50 as primary antibody; Lanes 8,9,10: Immunostaining using anti-actin as primary antibody. Lanes 2,5,8: actin Lanes 3,6,9: hrtrRNASET2-50 (SEQ ID NO: 14) Lanes 4,7,10: actin-hrtrRNASET2 complex;

FIG. 5B is a graphic representation of hrtrRNASE T2 actin binding in a solid-phase ELISA immunoassay. Each well of a 96-microtiter plate was coated with G-actin (500 ng/100 μl/well) in 50 mM buffer carbonate pH 9.5 for 1 hour and then washed with TBS. The coated wells were blocked with 3% BSA in 200 μl TBS for 1 hour and then washed with TBS. hrtrRNASET2-actin binding was performed using 1:2 serial dilution, starting from 125 ng/well of the protein in 100 μl TBS for 1 hour. The wells were washed three times with TBST and incubated with polyclonal rabbit anti-hrtrRNASET2-50 antibody (1:500 in 100 μl/well TBS for 1 hour followed by three washes with TBST) and then with goat anti-rabbit-HRP conjugated (1:10,000 in 100 μl/well TBS for 1 hour followed by three washes with TBST). Actin binding was detected using 1-Step™ Ultra TMB-ELISA, measuring absorbance at 655 nm;

FIGS. 6A and 6B depict the effect of truncated forms of RNASET2 (1 μM each) on clonogenicity in colon cancer HT29 cells. FIG. 6A is a histogram illustrating the results of HT29 cells cultured in medium in the presence of hrtrRNASE T2-50, hrtrRNASE T2-70, Mock or without added protein (PBS). Note that the number of colonies (percent of control) is significantly lower in hrtrRNASE T2-50 (SEQ ID NO: 14) and in hrtrRNASE T2-70 (SEQ ID NO: 15) (P<0.01) relative to control and Mock (N=5 for each treatment). FIG. 6B is a microphotograph showing growth of HT29 colonies in the presence of hrtrRNASE T2-50 (SEQ ID NO: 14), hrtrRNASE T2-70 (SEQ ID NO: 15), control and Mock. Cells were grown 5 days, fixed in formaldehyde and stained with methylene blue;

FIGS. 7A-7L are photographs illustrating the effect of human truncated RNASET2 on in-vitro HUVEC tube formation in the Matrigel™ assay. Tube formation of HUVEC on Matrigel™ in a 96-well microtiter plate (14×10³ cells/well) was induced by angiogenin (FIGS. 7A-7D), bFGF (FIGS. 7E-7H) or VEGF (FIGS. 7I-7L), 1 μg/ml each). In addition, the cells were treated with hrtrRNASE T2-50 (SEQ ID NO: 14)(7D, 7H and 7L), hrtrRNASE T2-70 (SEQ ID NO: 15)(7C, 7G and 7K) or insert-less vector extract (Mock, 7B, 7F and 7J) (200 μg/ml of each protein), or PBS (Control, 7A, 7E and 7I). Note that in wells treated by PBS or Mock, HUVEC tube formation is evident; however in wells exposed to hrtrRNASRE2-50 (SEQ ID NO: 14) or hrtrRNASRE2-70 (SEQ ID NO: 15), tube formation is inhibited. (N=5 for each treatment);

FIGS. 8A-8O are photographs illustrating the effect of different doses of hrtrRNASET2-50 (SEQ ID NO: 14) on in-vitro HUVEC tube formation in the Matrigel™ assay. The experiment was done as described in FIG. 7, using hrtrRNASET2-50 at concentrations of 0.5 (8D-8F), 2.5 (8G-8I), 5 (8J-8L) and 10 (8M-8O) μM, and growth factors angiogenin (8A, 8D, 8G, 8J and 8M), bFGF (8B, 8E, 8H, 8K and 8N) and VEGF (8C, 8F, 8I, 8L and 8O) (200 μg/ml of each protein). PBS was used as control (8A-8C). In all growth factors, hrtrRNASET2-50 inhibited HUVEC tube formation at a dose-responsive manner. In angiogenin, in-vitro tube formation was inhibited at 0.5 μM hrtrRNASET2-50. In bFGF and VEGF, in-vitro inhibition was observed at 2.5 μM hrtrRNASET2-50;

FIG. 9 is a graph illustrating in-vivo inhibition of tumor growth by hrtrRNASE T2-50 (SEQ ID NO: 14). The effect of systemic administration of 5 mg/kg hrtrRNASE T2-50 on tumor growth of HT-29-derived colon cancer cells implanted in nude mice was expressed as Relative Tumor Volume (Relative Tumor Volume (RTV)=Vᵢ/V₀, where Vᵢ is the tumor volume at any given time and V₀ is that at the time of initial treatment), measured over 5 weeks post-implantation. Each bar represents the standard error of the mean;

FIGS. 10A-10F are photographs of histological sections illustrating the in-vivo inhibition of tumor growth by systemic hrtrRNASE T2-50 (SEQ ID NO: 14) administration. HT-29-derived xenografts were grown in nude mice, fixed in paraffin and sectioned after hematoxylinand eosin staining. (Mock)-treated tumors (10C, 10D), hrtrRNASE T2-50 (SEQ ID NO: 14)-treated tumors (10E, 10F). In control (10A) and insert-less vector extract (Mock)-treated mice (10C), low-magnification observation reveals broad areas of invasive growth of the HT-29 cancer cells, accompanied by extensive angiogenic growth. High magnification of the blood vessels shows the cancer cells extending into the endothelial cells (10B, 10D). In mice treated systemically with hrtrRNASE T2-50 (SEQ ID NO: 14), low magnification observation (10E) reveals cancer cells concentrated in clusters surrounded by necrotic tissue, and decreased angiogenesis. High magnification shows tumor cells detached from the blood vessel and destruction of the endothelial structure (10F). Scale bar=75 μm (10A, 10C, 10E); 15 μm (10B, 10D, 10F);

FIG. 11 is a graphic representation showing the in-vivo effect of systemic hrtrRNASE T2-70 (SEQ ID NO: 15) administration on HT-29-derived colon cancer cells implanted in nude mice. Following cancer cell implantation and establishment of a tumor, mice received of 5 mg/kg hrtrRNASE T2-70 (solid triangles ▴), insert-less vector extract (Mock, solid squares ▪) or no protein (PBS, solid circles ). Each bar represents the standard error of the mean. Relative Tumor Volume (RTV)=Vᵢ/V₀, where Vᵢ is the tumor volume at any given time and V₀ is that at the time of initial treatment (Fujii T et al. Cancer Research (2003), 23: 2405-2412).

### DESCRIPTION OF EMBODIMENTS OF THE INVENTION

The invention described herein, in some embodiments thereof, relates to methods for efficient bacterial expression of truncated human T2 RNase having anti-tumor and anti-angiogenic properties, and further, to the therapeutic use of the recombinant truncated human T2 RNase.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Recombinant human RNASET2, produced in the P. pastoris expression system, has been shown to have significant tumor suppressive and anti-angiogenic effects, which are not mediated by its ribonuclease activity (see WO 2006/035439, which is hereby incorporated in its entirety).

In order to increase the expression levels and to avoid glycosylation of the expressed protein, hrRNASET2 was expressed in E. coli.

The present inventors found that expression of the full length RNASET2 gene in E. coli resulted in trace amounts, or in none of the recombinant protein. To remedy this, two truncated versions of the gene, encoding for RNASET2 peptides starting at Glu50 (hrtrRNASET2-50)(SEQ ID NO: 2) and at Met70 (hrtrRNASET2-70)(SEQ ID NO: 3) were constructed and expressed in E. coli (SEQ ID NOs: 14 and 15, respectively) These truncated human recombinant RNASET2 proteins retained the therapeutic and actin-binding properties of the A. niger B1 fungal T2 RNase and the full-length, yeast-produced human RNASET2.

Thus, according to one aspect of the present invention there is provided an isolated human truncated RNASET2. The RNASET2 is devoid of ribonucleolytic activity and has anti-angiogenic activity.

As used herein, the term “RNASET2” relates to the human member of the T2 family of RNases, previously known as “RNase6P1” or “human T2 RNase”. “RNASET2”(SEQ ID NO:1) is encoded by the RNASET2 gene, located at the 6q27 region of the human genome (see Campomenosi et al, Arch Biochem Biophys, 2006; 449:17-26).

As used herein, the term “isolated” refers to a protein or polypeptide removed from its normal physiological context.

As used herein, the term “truncated” refers to a RNASET2 protein or polypeptide which is missing a number of amino acids, usually missing a portion of the polypeptide chain. A truncated protein can be truncated (missing a portion of the polypeptide chain) at the N-terminal or C-terminal regions, or at any point (or points) therebetween.

The human truncated RNASET2 of the present invention can be truncated in any region which results in a RNASET2 polypeptide devoid of ribonucleolytic activity yet retaining anti-angiogenic properties. It will be noted that human RNASET2 comprises 4 pairs (eight altogether) of cysteine residues, at amino acid coordinates 25, 32, 52, 98, 161, 179, 190 and 208, possibly related to functional properties of the polypeptide. According to one embodiment, the human truncated RNASET2 is truncated in the putative N-terminal ribonuclease catalytic domain. According to another embodiment, the human truncated RNASET2 is devoid of the amino acid sequence corresponding to amino acid residues 1-32 of the N-terminus of SEQ ID NO: 1 (full-length RNASET2), thus devoid of the cysteine residues at amino acid coordinates 25 and 32. According to still another embodiment, the human truncated RNASET2 is devoid of the amino acid sequence corresponding to amino acid residues 1-52 of the N-terminus of SEQ ID NO: 1 (full-length RNASET2), thus devoid of the cysteine residues at coordinates 25, 32 and 52. According to another embodiment, the human truncated RNASET2 is devoid of the amino acid sequence corresponding to amino acid residues 1-49 of the N-terminus of full-length RNASET2. According to still another embodiment, the human truncated RNASET2 is devoid of the amino acid sequence corresponding to amino acid residues 1-69 of the N-terminus of SEQ ID NO: 1. Exemplary human truncated RNASET2 proteins are shown in SEQ ID NOs: 2 and 3. According to yet another embodiment, the human truncated RNASET2 is a fusion protein, further comprising a recognition entity peptide (e.g. His-tag) at the C-terminal or N-terminal ends of the polypeptide. Optionally, in another embodiment, the human truncated RNASET2 fusion protein further comprises a peptide linker located between the recognition entity peptide and the RNASET2 amino acid sequence, for example, a protease cleavage site (e.g. exokinase cleavage site, thrombin cleavage site, and the like). Exemplary human truncated RNASET2 fusion proteins having a recognition entity peptide and a protease cleavage site linker are shown in SEQ ID NOs: 14 and 15.

Thus, in one embodiment, the truncated RNASET2 protein devoid of ribonucleolytic activity and having anti-angiogenic activity is at least 75%, at least 80%, at least 85%, preferably 88%, more preferably 90%, yet more preferably 93%, still more preferably 95%, yet more preferably 98%, and most preferably 100% homologous to SEQ ID NOs: 2 or 3. In a yet further embodiment, the RNASET2 protein is as set forth in SEQ ID NOs: 2, 3, 14 and 15.

The ribonucleases of the T2 family have been identified in numerous microorganisms, as well as in plant and animal species, and are characterized by their unique molecular features (for a detailed review of T2 RNases, see Deshpande et al, Crit Rev Microbiol, 2002; 28:79-122 and WO 2006/035439, which is fully incorporated herein by reference). It will be appreciated that non-human T2 RNase, having anti-angiogenic, anti-tumor and anti-metastatic properties can also be truncated as described herein, to eliminate ribonucleolytic activity and enhance recombinant expression in bacteria and other expression systems.

As used herein, the term “angiogenesis” refers to the de novo formation of vessels such as that arising from vasculogenesis as well as those arising from branching and sprouting of existing vessels, capillaries and venules. Angiogenesis can be assessed, for example, by histological analysis of a tissue sample, by monitoring expression of typical angiogenesis-related genes (e.g. endothelial-specific genes), and by in-vitro assays such as the HUVE cell (HUVEC)-Matrigel™ assay described in detail hereinbelow. “Tumor angiogenesis” refers to the formation of blood vessels associated with tumor growth. “Anti-angiogenesis” refers to inhibition, reduction, prevention or limitation of angiogenic processes.

As used herein, the term “ribonucleolytic activity” refers to both endoribonuclease activity and exoribonuclease activity. Truncated RNASET2 “devoid of ribonucleolytic activity” refers to a RNASET2 essentially lacking ribonuclease activity, although traces of residual RNase activity may be detected when assayed.

According to one embodiment, the human truncated RNASET2 has actin-binding activity. According to yet another embodiment, the actin-binding activity of the T2 RNase is thermostable. Some therapeutic properties of T2 RNases have been correlated with actin-binding (see WO 2006/035439). Without limiting the present invention by any theory, it is believed that an ability of a T2 ribonuclease to bind to actin is indicative that such a T2 ribonuclease has anti-proliferation, anti-angiogenic and anti-tumor activities.

Actin binding can be assessed by a variety of assays, including but not limited to solution binding assays (e.g. the EDC assay detailed herein), PAGE separation and Western blotting, filter-based assays and ELISA-based assays (as detailed herein). Actin binding assay kits are commercially available, for example, from Cytoskeleton, Inc. (Denver, Colo., USA).

The human truncated RNASET2 protein can be recombinantly produced by expressing a polynucleotide encoding same, using an appropriate expression vector system. Thus, according to one embodiment there is provided an isolated polynucleotide encoding a human truncated RNASET2, wherein said human truncated RNASET2 is devoid of ribonucleolytic activity and has anti-angiogenic activity.

Exemplary polynucleotides encoding the human truncated RNASET2 devoid of ribonucleolytic activity and having anti-angiogenic activity include, but are not limited to polynucleotides encoding hrtrRNASET2-50 and hrtrRNASET2-70. Thus, in one embodiment, the polynucleotide is at least 75%, at least 80%, at least 85%, preferably 88%, more preferably 90%, yet more preferably 93%, still more preferably 95%, yet more preferably 98%, and most preferably 100% homologous to SEQ ID NOs: 4 or 5. In a yet further embodiment, the polynucleotide is as set forth in SEQ ID NOs: 4, 5, 12 or 13.

As such, the term “polynucleotide” when used herein in context of truncated RNASET2 in general, or in context of any specific truncated RNASET2, refers to any polynucleotide sequence which encodes a RNASET2 polypeptide active in preventing, inhibiting and/or reversing angiogenesis and devoid of ribonucleolytic activity.

The term “nucleic acid” refers to polynucleotides or to ologonucleotides such as deoxyribonucleic acid (DNA), and, where appropriate, ribonucleic acid (RNA) or mimetics thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, enhanced cellular uptake, enhanced affinity for nucleic acid target and increased stability in the presence of nucleases.

DNA encoding the human truncated RNASET2 is readily isolated and sequenced using conventional procedures. Once isolated, the DNA can be ligated into expression vectors, which are then transfected into bacterial host cells.

The DNA sequence encoding the human truncated RNASET2 is inserted into a recombinant vector which may be any vector, which may conveniently be subjected to recombinant DNA procedures, and the choice of vector will often depend on the host cell into which it is to be introduced.

In one embodiment, the expression system is a bacterial heterologous expression system. Suitable expression vector systems include bacteria transformed with bacteriophage DNA, plasmid DNA, or cosmid DNA. The expression controlling elements of vectors vary in their strengths and specifications depending on the host-vector system utilized, any one of a number of suitable transcription and translation elements may be used.

The vector components generally include, but are not limited to, one or more of the following: a promoter, an origin of replication, one or more selection markers, and a transcription terminator sequence. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the RNASET2 polypeptide is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The bacterial host is selected capable of producing the recombinant proteins (i.e., RNASET2) as inclusion bodies (i.e., nuclear or cytoplasmic aggregates of stainable substances).

According to specific embodiments of the present invention the host cells are selected from Gram-negative or Gram-positive bacterium/bacteria. Examples of Gram-negative bacteria which can be used in accordance with the present teachings include, but are not limited to, Escherichia coli, Pseudomonas, erwinia and Serratia. Examples of Gram-positive bacteria which can be used in accordance with the present teachings include, but are not limited to bacteria of genus Enterococcus, Melissococcus, Peptococcus, Saccharococcus, Staphylococcus, Streptococcus and Vagococcus. Choice of host will be made with consideration of cost of operation and optimizing cell culture densities, to provide highest product yields at reasonable expense.

The procedures used to ligate the DNA sequences coding for the polypeptides, the promoter (e.g., constitutive or inducible) and optionally the terminator, recognition entity peptide and/or protease cleavage site sequences, respectively, and to insert them into suitable vectors containing the information necessary for replication, are well known to persons skilled in the art (see, for instance, Sambrook et al., Molecular Cloning: A Laboratory Manual, Cold Spring Harbor, N.Y., 1989). Exemplary polynucleotide sequences encoding truncated human RNASET2 having a protease cleavage site are shown in SEQ ID NOs: 12 and 13.

Examples of bacterial expression vectors suitable for use in accordance with the present teachings include, but are not limited to, pET™ systems, the T7 systems and the pBAD™ system, which are well known in the art. In one embodiment, the bacterial expression vector is pHis3Parallel, a pET-based vector optimized for expression of fusion proteins having a His tag recognition entity peptide sequence (Sheffield, et al., Prot Expr Purif. 1999; 15:34-39). In another embodiment, a protease cleavage site sequence can be included, in order to facilitate removal of the His tag recognition sequence following purification of the protein. A non-limiting list of proteases which have well defined cleavage sites suitable for use in His tag removal includes enterokinase (light chain, available from New England Biolabs, MA, USA), thrombin (available from Novagen, Inc., WI, USA), HRV 3C protease (available from Novagen, Inc., WI, USA) and tobacco etch virus (TEV) (available from Nacalai USA, San Diego, Calif.). Optionally, solubility domains, well known in the art, can also be included to aid in the recovery of recombinant proteins.

Methods of introducing expression vectors into bacterial host cells are well known in the art and mainly depend on the host system used. These include, but are not limited to, electroporation, chemical transformation, conjugation, transduction, and the like. Recombinant DNAs can be easily introduced into those that are naturally competent by transformation.

Host cells are cultured under effective conditions, which allow for the expression of high amounts of human truncated RNASET2. Effective culture conditions include, but are not limited to, effective media, bioreactor, temperature, pH and oxygen conditions that permit recombinant protein production. An effective medium refers to any medium in which a bacterium is cultured to produce the recombinant protein of the present invention. Such a medium typically includes an aqueous solution having assimilable carbon, nitrogen and phosphate sources, and appropriate salts, minerals, metals and other nutrients, such as vitamins. Bacterial hosts of the present invention can be cultured in conventional fermentation bioreactors, shake flasks, test tubes, microtiter dishes, and petri plates, dependent on the desired amount. Culturing can be carried out at a temperature, pH and oxygen content appropriate for a recombinant host. Such culturing conditions are within the expertise of one of ordinary skill in the art.

Once appropriate expression levels of recombinant truncated RNASET2 are obtained the polypeptides are recovered from the inclusion bodies. Methods of recovering recombinant proteins from bacterial inclusion bodies are well known in the art and typically involve cell lysis followed by solubilization in denaturant [e.g., De Bernardez-Clark and Georgiou, “Inclusion bodies and recovery of proteins from the aggregated state” Protein Refolding Chapter 1:1-20 (1991). See also Examples section which follows, Example I: “Cloning, Expression and Purification of hrtrRNASET2-50 (SEQ ID NO: 2) and hrtrRNASET2-70 (SEQ ID NO: 3)].

Briefly, the inclusion bodies can be separated from the bulk of cytoplasmic proteins by simple centrifugation giving an effective purification strategy. They can then be solubilized by strong denaturing agents like urea (e.g., 8 M) or guanidinium hydrochloride and sometimes with extremes of pH or temperature. The denaturant concentration, time and temperature of exposure should be standardized for each protein. Before complete solubilization, inclusion bodies can be washed with diluted solutions of denaturant and detergent to remove some of the contaminating proteins.

Finally, the solubilized inclusion bodies can be directly subjected to further purification through chromatographic techniques prior to or following removal of denaturing agents. Exemplary methods for recovering, separating and purifying a protein are detailed hereinbelow.

Separation of truncated RNASET2 can be performed to purify the polypeptide from proteins and other components of the bacteria and culture medium. Purification of recombinant proteins is particularly important and desirable for, for example, therapeutic applications. Thus, according to one embodiment, there is provided a purified preparation of human truncated RNASET2 having anti-angiogenic properties and devoid of ribonucleolytic activity. RNASET2 polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing, size filtration and differential solubilization. In one embodiment, the truncated RNASET2 comprises a recognition entity peptide sequence, and purification is performed by affinity chromatography in order to isolate the desired, recognition entity-bearing polypeptide from the proteins of, for example, a bacterial lysate. Recognition entity peptides can be optionally engineered at either the amino or carboxy terminal regions of the recombinant protein. Useful recognition entity sequences include, but are not limited to, a polyhistidine tract (HHHHHH), the IgG binding domain of protein A, glutathione S-transferase (GST), calmodulin binding peptide, biotin and the like. Recombinant proteins can be easily purified in a one step process using, for example, metal chelation (e.g., Ni-agarose), protein A-Sepharose, and glutathione-Sepharose column chromatography. N-terminal or C-terminal signal and recognition entity sequences can be easily removed by incorporating a protease cleavage site.

Thus, according to one embodiment of the present invention, the recognition entity is a consecutive stretch of 6 to 10 histidine residues (HHHHHH). A polyhistidine sequence of six amino acid residues has been shown to be poorly immunogenic and rarely affects protein function and structure. The polyhistidine recognition entity peptide can be engineered at either the amino or carboxy terminus of the protein. Thus, in another embodiment, the recognition entity peptide sequence is a His-tag, and purification is performed by reversible Nickel-Histidine binding to a Nickel affinity medium, as described in detail in Example I herein. The use of recognition entity peptides in the form of poly-histidine residues (socalled “His-tag”) C- or N-terminally fused to a protein, for the purification and/or for functional studies of proteins has been described (Janknecht et al., Proc. Natl. Acad. Sci. USA 88:8972-8976, 1991, Hoffmann et al., Nucleic Acids Res 19:6337-6338, 1991, EP 0 282 042).

In one embodiment, following purification the truncated RNASET2 protein remains with the recognition-entity sequence intact (for example, His tag), and is used as a fusion protein. In another embodiment, and optionally, the recombinant RNASET2 is expressed including a protease cleavage site adjacent to the recognition-entity peptide, and following purification the recombinant RNASET2-cleavage site-recognition entity peptide is treated with a protease, or combination of proteases, to remove the recognition-entity sequence from the purified recombinant RNASET2. Suitable cleavage sites are well known in the art, and include, but are not limited to, the enterokinase cleavage site, thrombin cleavage site, HRV 3C protease cleavage site, tobacco etch virus (TEV) cleavage site. The construction and use of such protease cleavage sites in expression and purification of recombinant proteins is well known in the art. In a further embodiment, the recognition entity peptide is removed using an exopeptidase or a combination of exopeptidases, such as dipeptidyl aminopeptidase, glutamine cyclotransferase and pyroglutamyl aminopeptidase (TAGZyme, Qiagene, CA, USA) and the like. Exemplary truncated human RNASET2 polypeptides having protease cleavage site linkers and recognition entity peptides are shown in SEQ ID NOs: 14 and 15.

The above-described methodology is efficient for obtaining unprecedented yields of highly purified recombinant human RNASET2 having anti-angiogenic and anti-tumor activity from prokaryotic cells. Accurate expression of the RNASET2 proteins can be examined functionally and structurally. Methods of assaying activity are described at length in the Examples section which follows (e.g., PAGE separation, actin-binding assays, immunodetection, in-vitro and in-vivo angiogenesis assays, antigenic recognition).

The present teachings provide truncated human RNASET2 devoid of ribonucleolytic activity and having anti-angiogenic activity in a yield of at least 50 mg, optionally at least 75 mg, optionally at least 100 mg, optionally at least 120 mg, optionally at least 150 mg, optionally at least 200 mg, optionally at least 300 mg, optionally at least 500 mg, optionally at least 750 mg, and optionally at least 1000 mg of purified human truncated RNASET2 molecules per 1 liter of bacterial culture at the time of induction.

Thus, embodiments of the present invention provide for a composition-of-matter comprising bacterial preparation remnants and at least about 70%, 80%, 85%, 90%, 95% or more human truncated RNASET2. Bacterial remnants may be further removed for clinical applications (in vivo) using methods which are well known in the art.

A truncated human RNASET2 can be used to prepare a medicament according to the present invention by means of conventional mixing, dissolving, granulating, dragee-making, levigating, emulsifying, encapsulating, entrapping or lyophilizing processes with the addition of the appropriate pharmaceutically acceptable carriers and/or excipients or alternatively it can be linked to appropriate delivery vehicles as described hereinabove.

While reducing the present invention to practice, it was shown, that the truncated human RNASET2 effectively inhibits tumor growth, metastatic proliferation and angiogenesis in-vitro and in-vivo (see Examples III-IV). Thus, the truncated RNASET2 of the present invention, and compositions comprising such, may be used as therapeutic agents for controlling cellular disorders related to motility, including cancer (e.g. tumor angiogenesis and metastasis), immune regulation, neurodegenerative and inflammatory disease.

Thus, according to one embodiment of the present invention there is provided a method of inhibiting angiogenesis in a subject in need thereof. The method is effected by providing a truncated human RNASET2 protein having anti-angiogenic activity, preferably having an amino acid sequence at least 95% homologous to SEQ ID NOs: 2 or 3.

Truncated human RNASET2 was shown to specifically bind to actin. Disruption of actin assembly and disassembly affects cell motility, development, growth, proliferation and reproduction. Thus, the compositions and methods of present invention can be used for treating conditions, syndromes or diseases characterized by abnormal accumulation of cells. Diseases or conditions characterized by abnormal accumulation of cells include, but are not limited to, inflammatory diseases, neurodegenerative diseases, and cancer. Further, the compositions and methods of the present invention can be used for inhibiting actin filament assembly and disassembly in a cell or a tissue, effected by providing to the cell or tissue a truncated human RNASET2 protein, for example, RNASET2-50 or RNASET2-70 or a truncated homologues thereof having anti-angiogenic and anti-tumor activity.

Thus, the present invention can be used for treating conditions, syndromes or diseases characterized by abnormally proliferating cells, such as cancerous or other cells, such as, but not limited to, a malignant or non-malignant cancer including biliary tract cancer; brain cancer; breast cancer; cervical cancer; choriocarcinoma; endometrial cancer; esophageal cancer; gastric cancer; intraepithelial neoplasms; lymphomas; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas, papilloma, blastoglioma, Kaposi's sarcoma, squamous cell carcinoma, astrocytoma, head cancer, neck cancer, bladder cancer, colorectal cancer, thyroid cancer, pancreatic cancer, gastric cancer, hepatocellular carcinoma, leukemia, lymphoma, Hodgkin's disease, Burkitt's disease, arthritis, rheumatoid arthritis, diabetic retinopathy, angiogenesis, restenosis, in-stent restenosis, vascular graft restenosis, proliferative vitreoretinopathy, chronic inflammatory proliferative disease, dermatofibroma and psoriasis.

As used herein the terms “cancer” or “tumor” are clinically descriptive terms which encompass a myriad of diseases characterized by cells that exhibit abnormal cellular proliferation. The term “tumor”, when applied to tissue, generally refers to any abnormal tissue growth, characterized in excessive and abnormal cellular proliferation. A tumor may be “benign” and unable to spread from its original focus, or “malignant” or “metastatic” and capable of spreading beyond its anatomical site to other areas throughout the host body. The tumor may be a “primary” tumor, residing in the organ in which it has developed, and which is not a metastatic growth, or it may be a metastatic tumor, developing in an organ other than that of the primary tumor. The term “cancer” is an older term which is generally used to describe a malignant tumor or the disease state arising therefrom. Alternatively, the art refers to an abnormal growth as a neoplasm, and to a malignant abnormal growth as a malignant neoplasm.

The truncated human RNASET2 of the present invention can be used in the preventive treatment of a subject at risk of having a cancer. A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is exposed to the RNASET2 of the present invention, the subject may be able to prevent any cancer that does form from becoming metastatic.

The RNASET2 of the present invention is also useful for treating and/or preventing disorders associated with inflammation in a subject. Immune or hematopoietic cells exposed to RNASET2 having an actin binding activity would have a reduced ability to migrate. Thus RNASET2 having actin binding activity is useful for preventing inflammation associated with immune cell migration and for treating and preventing inflammatory disorders and ischemic diseases.

Inflammatory disorders and ischemic diseases are characterized by inflammation associated with neutrophil migration to local tissue regions that have been damaged or have otherwise induced neutrophil migration and activation. While not intending to be bound by any particular theory, it is believed that excessive accumulation of neutrophils resulting from neutrophil migration to the site of injury, causes the release toxic factors that damage surrounding tissue. When the inflammatory disease is an acute stroke a tissue which is often damaged by neutrophil stimulation is the brain. As the active neutrophils accumulate in the brain an infarct develops.

An “inflammatory disease or condition” as used herein refers to any condition characterized by local inflammation at a site of injury or infection and includes autoimmune diseases, certain forms of infectious inflammatory states, undesirable neutrophil activity characteristic of organ transplants or other implants and virtually any other condition characterized by unwanted neutrophil accumulation at a local tissue site. These conditions include but are not limited to meningitis, cerebral edema, arthritis, nephritis, adult respiratory distress syndrome, pancreatitis, myositis, neuritis, connective tissue diseases, phlebitis, arteritis, vasculitis, allergy, anaphylaxis, ehrlichiosis, gout, organ transplants and/or ulcerative colitis.

An “ischemic disease or condition” as used herein refers to a condition characterized by local inflammation resulting from an interruption in the blood supply to a tissue due to a blockage or hemorrhage of the blood vessel responsible for supplying blood to the tissue such as is seen for myocardial or cerebral infarction. A cerebral ischemic attack or cerebral ischemia is a form of ischemic condition in which the blood supply to the brain is blocked. This interruption in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue.

In some aspects of the invention the RNASET2 of the present invention is provided in an effective amount to prevent migration of a tumor cell across a barrier. The invasion and metastasis of cancer is a complex process which involves changes in cell adhesion properties which allow a transformed cell to invade and migrate through the extracellular matrix (ECM) and acquire anchorage-independent growth properties (Liotta, L. A., et al., Cell 1991 64:327-336). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

In yet another embodiment, the RNASET2 of the present invention can be used to assay cells for sensitivity to inhibition of cellular motility, for example, in testing their ability to cross a barrier. Preferably the tumor cells are prevented from crossing a barrier. The barrier for the tumor cells may be an artificial barrier in vitro or a natural barrier in vivo. In vitro barriers include but are not limited to extracellular matrix coated membranes, such as Matrigel™. Thus, RNASET2 can be provided to cells which can then be tested for their ability to inhibit tumor cell invasion in a Matrigel invasion assay system. Other in vitro and in vivo assays for metastasis have been described in the prior art, see, e.g., U.S. Pat. No. 5,935,850, which is incorporated herein by reference. An in vivo barrier refers to a cellular barrier present in the body of a subject.

The truncated human RNASET2 according to one aspect of the present invention can be administered to an organism, such as a human being or any other mammal, per se, or in a pharmaceutical composition where it is mixed with suitable carriers or excipients.

As used herein a “pharmaceutical composition” or “medicament” refers to a preparation of one or more of the truncated human RNASET2 ribonucleases as described herein, with other chemical components such as physiologically suitable carriers and excipients. The purpose of a pharmaceutical composition is to facilitate administration of a compound to an organism.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Pharmaceutical compositions may also include one or more additional active ingredients, such as, but not limited to, anti inflammatory agents, antimicrobial agents, anesthetics, cancer therapeutic agents and the like in addition to the main active ingredient. A detailed description of commonly used additional agents suitable for use with the compositions of the present invention is presented hereinbelow.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which can be used pharmaceutically. Proper formulation is dependent upon the route of administration chosen.

Significant therapeutic effects of ribonucleases of the T2 family have been revealed using a broad variety of means of administration, in diverse models of abnormal cell proliferation and accumulation, angiogenesis, metastatic proliferation and tumor growth (see WO 2006/035439, fully incorporated herein by reference). Intraperitoneal administration, providing rapid systemic uptake and distribution of the RNase, was found effective in suppressing tumor growth and development in subcutaneous tumors in nude mice and intraperitoneal tumors. Intravenous administration, providing even more rapid systemic uptake of T2 RNase, was also found effective in suppressing and treating subcutaneous xenografts (see Example IV below), and remote (lung) metastatic spread of intravenous tumors. Direct administration of, and preincubation of cells with T2 RNase has been found effective in preventing tumor growth in breast carcinoma, colon carcinoma, melanoma in-vivo, angiogenic factor induced angiogenesis and microvessel density and cell tube formation in both plant and human HUVE cells in-vitro. Oral administration of T2 RNase, in the form of microcapsules, has been found effective in reducing tumor growth, proliferation, tumor size, tumor vascularization and the number of aberrant crypt foci when administered early in colon tumor (DMH model) induction. Similar oral administration of T2 RNase to animals harboring already well developed tumors reduced the degree of vascularization and malignancy of colon cancer tumors in rats, despite exposure of the RNase to digestive processes and low doses presumed delivered intraintestinally. It will be appreciated that encapsulation methods providing effective intestinal release of compositions are well known in the art, and use of such is expected to increase the effectiveness of oral administration of truncated human RNASET2 in cases of already established tumors.

Thus, to effect administration the pharmaceutical composition of the present invention includes a suitable pharmaceutical carrier and an effective amount of truncated human RNASET2 having anti-angiogenic activity, and is administered, for example, topically, intraocularly, parenterally, orally, intranasally, intravenously, intramuscularly, subcutaneously or by any other effective means via methods well known in the art.

For intravenous, intramuscular or subcutaneous injection, a truncated human RNASET2 may be formulated in aqueous solutions, preferably in physiologically compatible buffers such as Hank's solution, Ringer's solution, or physiological saline buffer. For example, a physiologically appropriate solution containing an effective amount of a truncated human RNASET2 can be administered systemically into the blood circulation to treat a cancer or tumor which cannot be directly reached or anatomically isolated. A physiologically appropriate solution containing an effective amount of a truncated human RNASET2 may be directly injected into a target cancer or tumor tissue by a needle in amounts effective to treat the tumor cells of the target tissue.

For transmucosal administration, penetrants appropriate to the barrier to be permeated are used in the formulation. Such penetrants are generally known in the art.

For oral administration, the pharmaceutical composition of the present invention can be formulated readily by combining a truncated human RNASET2 with pharmaceutically acceptable carriers well known in the art. Such carriers enable a truncated human RNASET2 to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Additional pharmaceutical compositions, which can be used orally, include push-fit capsules made of gelatin as well as soft, sealed capsules made of gelatin and a plasticizer, such as glycerol or sorbitol. The push-fit capsules may contain a truncated human RNASET2 in admixture with filler such as lactose, binders such as starches, lubricants such as talc or magnesium stearate and, optionally, stabilizers. In soft capsules, a truncated human RNASET2 may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Oral delivery of the pharmaceutical composition of the present invention may not be successful due to the pH and enzyme degradation present in the gastrointestinal tract. Thus, such pharmaceutical compositions must be formulated to avoid undesirable circumstances. For example, enteric coating can be applied to oral solid formulation. Substances with acidic-resistant properties such as cellulose acetate phtalate (CAP), hydroxypropyl methycellulose phtalate (HPMCP) and acrylic resins are most commonly used for coating tablets or granules for micro encapsulation. Preferably wet granulation is used to prepare the enteric-coated granules to avoid reactions between the active ingredient and the coating (Lin, S. Y. and Kawashima, Y. 1987, Pharmaceutical Res. 4:70-74). A solvent evaporation method can also be used. The solvent evaporation method was used to encapsulate insulin administered to diabetic rats to maintain blood glucose concentration (Lin, S. Y. et al., 1986, Biomater, Medicine Device, Artificial organ 13:187-201 and Lin, S. Y. et al., 1988, Biochemical Artificial Cells Artificial Organ 16:815-828). It was also used to encapsulate biological materials of high molecular weight such as vial antigen and concanavalin A (Maharaj, I. Et al. 1984, J. Phamac. Sci. 73:39-42).

For buccal administration, in one embodiment, the pharmaceutical composition of the present invention may take the form of tablets or lozenges formulated in conventional manner.

For administration by inhalation, a truncated human RNASET2 for use according to one embodiment of the present invention is conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of a truncated human RNASET2 and a suitable powder base such as lactose or starch.

According to another embodiment, the pharmaceutical composition of the present invention may also be formulated for parenteral administration, e.g., by bolus injection or continuous infusion. A composition for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of truncated human RNASET2 may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of a truncated human RNASET2 to allow for the preparation of highly concentrated solutions.

Alternatively, a truncated human RNASET2 may be in a powder form for constitution with a suitable vehicle, e.g., sterile, pyrogen-free water, before use.

The pharmaceutical composition of the present invention may also be formulated in rectal compositions such as suppositories or retention enemas, using, e.g., conventional suppository bases such as cocoa butter or other glycerides.

In addition, a cancer or tumor present in a body cavity, such as in the eye, gastrointestinal tract, genitourinary tract (e.g., the urinary bladder), pulmonary and bronchial system and the like, can receive a physiologically appropriate composition (e.g., a solution such as a saline or phosphate buffer, a suspension, or an emulsion, which is sterile) containing an effective amount of a truncated human RNASET2 via direct injection with a needle or via a catheter or other delivery tube placed into the cancer or tumor afflicted hollow organ. Any effective imaging device such as X-ray, sonogram, or fiber optic visualization system may be used to locate the target tissue and guide the needle or catheter tube in proximity thereto.

The pharmaceutical composition of the present invention can also be delivered by osmotic micro pumps. The osmotic micro pumps are implanted into one of the body cavities and the drug is constantly released onto the tissue to be treated. This method is particularly advantageous when an immune response to the pharmaceutical composition is experienced. This method has been employed for ONCONASE (Vasandani V. M., et al., 1996, Cancer Res. 15;56(18):4180-6).

Alternatively and according to yet another embodiment of the present invention, the pharmaceutically acceptable carrier includes a delivery vehicle capable of delivering a truncated human RNASET2 to the mammalian cell of the subject.

Numerous delivery vehicles and methods are known in the art for targeting proteins or nucleic acids into or onto tumors or cancer cells. For example, liposomes are artificial membrane vesicles that are available to deliver proteins or nucleic acids into target cells (Newton, A. C. and Huestis, W. H., Biochemistry, 1988, 27:4655-4659; Tanswell, A. K. et al., 1990, Biochmica et Biophysica Acta, 1044:269-274; and Ceccoll, J. et al., Journal of Investigative Dermatology, 1989, 93:190-194). Thus, a T2-RNase or a polynucleotide encoding same can be encapsulated at high efficiency with liposome vesicles and delivered into mammalian cells. In addition, the T2-RNase protein or nucleic acid can also be delivered to target tumor or cancer cells via micelles as described in, for example, U.S. Pat No. 5,925,628 to Lee, which is incorporated herein by reference.

Liposome or micelle encapsulated truncated human RNASET2 may be administered topically, intraocularly, parenterally, intranasally, intratracheally, intrabronchially, intramuscularly, subcutaneously or by any other effective means at a dose efficacious to treat the abnormally proliferating cells of the target tissue. The liposomes may be administered in any physiologically appropriate composition containing an effective amount of encapsulated truncated human RNASET2.

Alternatively and according to still another embodiment of the present invention the delivery vehicle can be, but it is not limited to, an antibody or a ligand capable of binding a specific cell surface receptor or marker. An antibody or ligand can be directly linked to a truncated human RNASET2 protein via a suitable linker, or alternatively such an antibody or ligand can be provided on the surface of a liposome encapsulating a truncated human RNASET2.

For example, a truncated human RNASET2 can be fused with specific membranal protein antibodies or ligands for targeting to specific tissues or cells as previously described in the art. It will be appreciated in this respect that fusion of RNase A of the ribonuclease A superfamily with antibodies to the transferrin receptor or to the T cell antigen CD5 lead to inhibition of protein synthesis in tumor cells carrying a specific receptor for each of the above toxins (Rybak, M. et al., 1991, J. Biol. Chem. 266:21202-21207 and Newton D L, et al., 1997, Protein Eng. 10(4):463-70).

Pharmaceutical compositions suitable for use in context of the present invention include compositions wherein the active ingredients are contained in an amount effective to achieve the intended purpose. More specifically, a therapeutically effective amount means an amount of the active ingredients effective to prevent, alleviate or ameliorate symptoms of disease or prolong the survival of the subject being treated.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Determination of a therapeutically effective amount is well within the capability of those skilled in the art, especially in light of the detailed disclosure provided herein.

Toxicity and therapeutic efficacy of the active ingredients described herein can be determined by standard pharmaceutical procedures in cell cultures or experimental animals, e.g., by determining the IC₅₀ and the LD₅₀ (lethal dose causing death in 50% of the tested animals) for a subject active ingredient. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can also be a single administration of a slow release composition, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to yet another aspect of the present invention there are provided methods of enhancing therapeutic treatment of a cancer. The methods are effected by administering to a subject in need thereof, in combination with the therapeutic treatment, a truncated human RNASET2. It will be appreciated that such synergistic activity of truncated human RNASET2 with additional therapeutic methods or compositions has the potential to significantly reduce the effective clinical doses of such treatments, thereby reducing the often devastating negative side effects and high cost of the treatment.

Therapeutic regimen for treatment of cancer suitable for combination with the truncated human RNASET2 of the present invention or polynucleotide encoding same include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

Anti-cancer drugs that can be co-administered with the compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's The Pharmacological Basis of Therapeutics“, Eighth Edition, 1990, McGraw-Hill, Inc. (Health Professions Division).

Anti-inflammatory drugs that can be administered in combination with the T2 RNase or polynucleotide encoding same of the present invention include but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

In yet another embodiment of the present invention, gene therapy with truncated human RNASET2 is envisaged. According to this aspect of the present invention a polynucleotide encoding a truncated human RNASET2 is introduced into a mammalian cell along with a pharmaceutically acceptable carrier, which introduction results in a genetic modification of this cell, enabling the expression of the truncated human RNASET2 therein.

Recently, Acquati et al have shown that transfection of RNase 6PL cDNA into HEY4 and SG10G ovarian tumor cell lines suppresses tumorigenicity in nude mice, and further that HEY4 clones and clones of a Xeroderma pigmentosum SV40-immortalized cell line, transfected with a RNase 6PL cDNA, develops a marked senescence process during in vitro growth (Aquati et al. Oncogene. 2001 22; 20(8):980-8), thus demonstrating the feasibility of such genetic modification with T2 RNase.

As used herein in the specification and in the claims section below, the term “genetic modification” refers to a process of inserting nucleic acids into cells. The insertion may, for example, be effected by viral infection, injection, transfection, particle bombardment or any other means effective in introducing nucleic acids into cells, some of which are further detailed hereinbelow. Following the genetic modification the nucleic acid is either integrated in all or part, to the cell's genome (DNA), or remains external to the cell's genome, thereby providing stably modified or transiently modified cells.

As used herein the phrases “gene therapy” or “genetic therapy” are used interchangeably and refer to a method of therapy in which a stable or transient genetic modification of a proliferative cell(s) such as a cancer cell, leads to the inhibition of proliferation of this cell. Any polynucleotides encoding truncated human RNASET2, for example SEQ ID NOs: 4, 5, 12 and 13 can be employed according to the present invention as a polynucleotide encoding truncated human RNASET2. In addition, polynucleotides homologous to SEQ ID NOs: 4, 5, 12 and 13 can also be employed as a polynucleotide encoding a truncated human RNASET2, provided that the protein encoded thereby is characterized as a truncated human RNASET2 and exhibits the desired anti-angiogenic activities. Furthermore, it will be appreciated that portions, mutants, chimeras or alleles of such polynucleotides can also be employed as a polynucleotide encoding a truncated human RNASET2 according to one embodiment of the present invention, again, provided that such portions, mutants chimeras or alleles of such polynucleotides encode a truncated human RNASET2 which exhibits the desired activities.

In another embodiment, a polynucleotide according to the present invention can be fused, in frame, to any other protein encoding polynucleotide to encode for a fused protein using methods well known in the art. In one embodiment, the polynucleotide encoding a truncated human RNASET2 is fused to a polynucleotide encoding a recognition entity peptide (e.g. His-tag). In yet a further embodiment, an optional polynucleotide sequence encoding a protease cleavage site (e.g. TEV cleavage site, enterokinase cleavage site, thrombin cleavage site, etc) is inserted in between the polynucleotide encoding a truncated human RNASET2 and the polynucleotide encoding the recognition entity peptide, encoding an RNASET2-cleavage site-recognition entity peptide fusion protein. The cleavage site and recognition entity sequences can be fused to the N-terminal or C-terminal region of the truncated human RNASET2 polypeptide.

A truncated human RNASET2 protein can be fused (conjugated) to other proteins using methods well known in the art. Many methods are known in the art to conjugate or fuse (couple) molecules of different types, including proteins. These methods can be used according to the present invention to couple a truncated human RNASET2 to other molecules such as ligands or antibodies to thereby assist in targeting and binding of the T2-RNase to specific cell types. Any pair of proteins can be conjugated or fused together using any conjugation method known to one skilled in the art. The proteins can be conjugated using a 3-(2-pyridyldithio)propionic acid N-hydroxysuccinimide ester (also called N-succinimidyl 3-(2pyridyldithio)propionate) (“SDPD”) (Sigma, Cat. No. P-3415), a gluteraldehyde conjugation procedure or a carbodiimide conjugation procedure.

Expression vectors compatible with mammalian host cells for use in genetic therapy of tumor or cancer cells, include, but are not limited to, plasmids, retroviral vectors, adenovirus vectors, herpes viral vectors, and non-replicative avipox viruses, as disclosed, for example, by U.S. Pat. No. 5,174,993.

Several methods can be used to deliver the expression vector according to this aspect of the present invention to the target mammalian cell(s).

According to yet another aspect of the present invention there is provided an anti-human truncated RNASET2 antibody, capable of specifically binding human truncated RNASET2. Preferably, the antibody specifically binds at least one epitope of a human truncated RNASET2. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). In one embodiment, the anti-human truncated RNASET2 antibody is a polyclonal antibody raised in rabbits against whole hrtrRNASET2-50 or hrtrRNASET2-70.

Human truncated RNASET2s and compositions (e.g., pharmaceutical composition) comprising same may be used in diagnostic and therapeutic applications and as such may be included in therapeutic or diagnostic kits.

Thus, compositions of the present invention may, if desired, be presented in a pack or dispenser device, such as an FDA approved kit, which may contain one or more unit dosage forms containing the active ingredient i.e., human truncated RNASET2. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, an and the include plural references unless the context clearly dictates otherwise. For example, the term “a compound” or at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1, 2, 317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

### Cloning, Expression and Purification of hrtrRNASET2-50 (SEQ ID NO: 2) and hrtrRNASET2-70 (SEQ ID NO: 3)

Constructs: The human RNASET2 gene was synthesized and optimized for E. coli by GENEART (SEQ ID NO: 11) to use as DNA template for the truncated forms of RNASET2. The truncated forms of the RNASE T2-50 coding sequence (cys6 573 bp, SEQ ID NO:4) and RNASE T2-70 coding sequence (cys5 513 bp, SEQ ID NO:5) were constructed by PCR with suitable primers for pHis3Parallel, and including linker sequences encoding an enterokinase cleavage site inserted between the His tag and RNASET2 sequence:

RNASE T2-50-Forward:
(SEQ ID NO: 7)
5′-CCATGGGTCACGATGATAAAATGCGCGCGTATTGGCCGGATG-3′
and
Reverse
(SEQ ID NO: 8)
5′-GCGGCCGCAAGCTTGGATCCTTAG-3′
RNASE T2-70-Forward:
(SEQ ID NO: 9)
5′-CCATGGGGTGACGATGATAAAGAAGGCTGTAATCGTAGCTGGCCGT
TC-3′
and
Reverse
(SEQ ID NO: 10)
5′-GCGGCCGCAAGCTTGGATCCTTAG-3′.

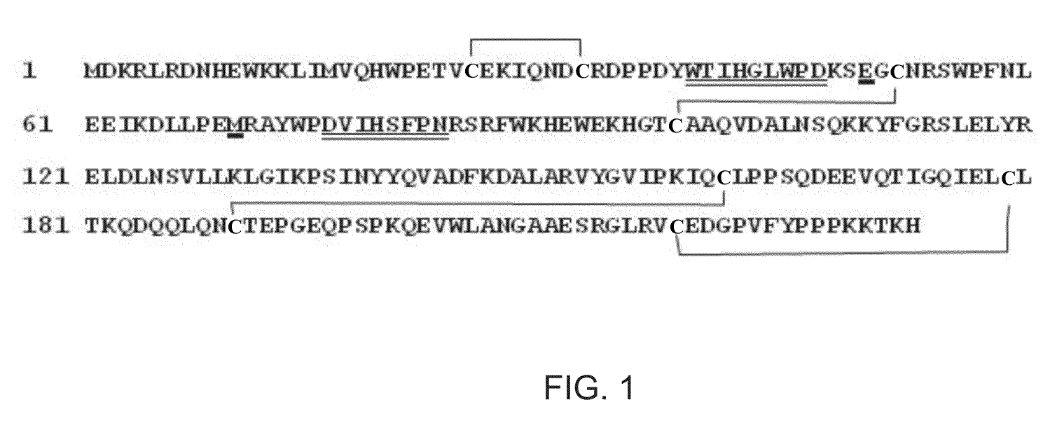

PCR mix contained 10 ng DNA template, dNTP mix (0.2 mM of each nucleotide), 0.4 pmol of each primer, 1 unite Imax Taq polymerase, 5 μl 10× Taq polymerase buffer and double distilled H₂0 to a final volume of 50 PCR was performed under the following conditions: denaturing at 94° C. for 2 min, 35 cycles of denaturing at 94° C. for 10 sec, annealing at 58° C. for 5 sec, elongation at 72° C. for 20 sec and than elongation at 72° C. for 4 min and termination at 10° C. for 10 min. The resulted amplified fragments of 573 bp and 513 bp were confirmed by sequencing and translation of the hrtrRNASET2-50 and hrtrRNASET2-70, respectively (FIG. 1).

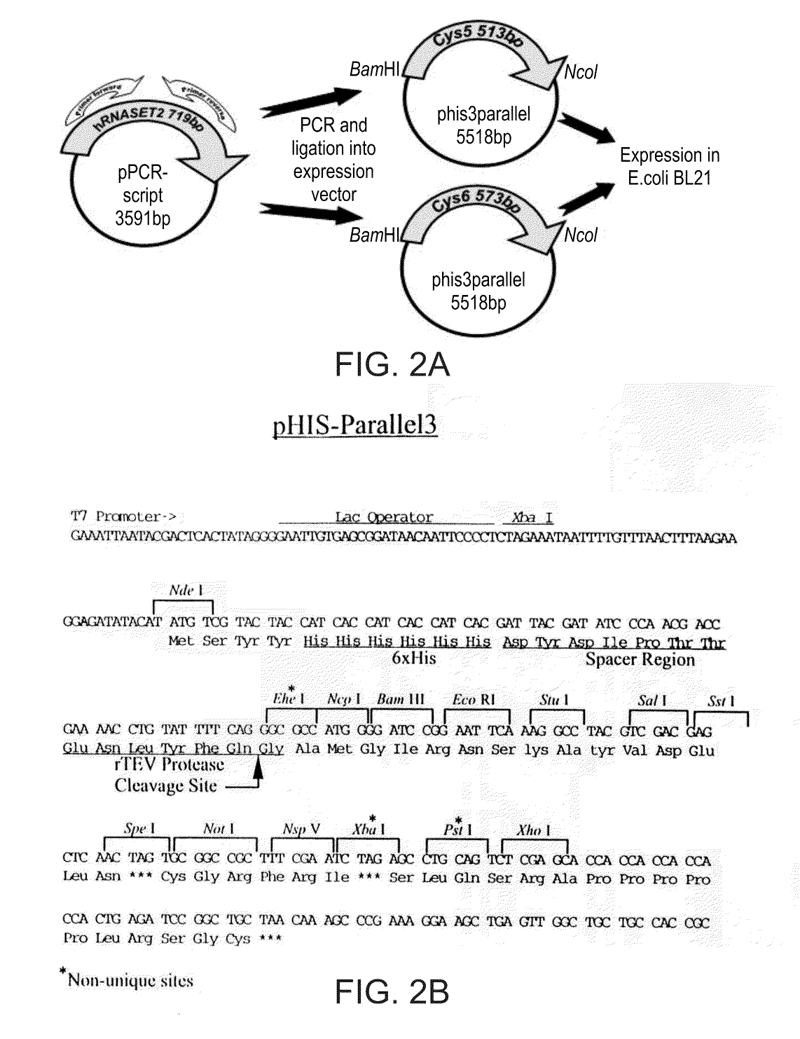

Transformations: The above PCR fragments were ligated into pHIS3Parallel vector (see FIG. 2B, SEQ ID NO: 6) using NcoI and BamHI as restriction enzymes and subcloned into E. coli DH5a for amplification. For protein expression, the vector containing each of the above inserts was cloned into E. coli BL21 (DE3) using the same procedure (FIG. 2). Insert-less “Mock” used the same pHis3Parallel vector without insert and was amplified in E. coli DH5α, then transformed into E. coli BL21 (DE3).

Bacterial Culture:

For protein expression, the transformed bacteria were grown in LB broth containing 100 μg/ml ampicillin at 37° C., 250 RPM until OD of 0.6-1 was obtained (2-3 hours). Expression of the recombinant protein was induced with 1 mM IPTG and incubation at the above conditions continued for 4 hours.

Harvest, Extraction and Purification of hrtrRNASET2:

The bacterial cells were centrifuged 10 min at 10,000 g. The pellet was kept at −80° C. until use. The cells were than lysed by resuspending the pellet in lysis buffer (20 mM Phosphate Buffer, 8 M urea, 100 mM NaCl, 1 mM EDTA pH 8.0) containing 2 mg/ml Complete Protease Inhibitor Cocktail (Roche Diagnostics, Mannheim, Germany) and stirring 2 h at 4° C. The cell debris was removed by centrifugation for 50 min at 12,000 g in 4° C. and the supernatant was filtered through a 0.2 μm filter.

The recombinant truncated proteins were purified using immobilized metal ion affinity chromatography (IMAC) using AKTAprime plus FPLC system (GE-Healthcare). The lysed baterial pellet was loaded onto 5-ml-HisTrap Ni column (GE-Healthcare) and eluted with imidasol gradient of 5-500 mM in equilibration buffer (20 mM sodium phosphate pH 8.0, 1 M NaCl, 8 M urea and 5 mM β-mercaptoethanol) at a flow rate of 5 ml/min. The His-tag and enterokinase cleavage sequences remained intact in the purified proteins. The collected fractions from the peak area were analyzed by SDS-PAGE.

Results:

Out of eight cysteine residues forming four disulfide bonds at the full RNASET2 sequence (Kurihara et al. 1992, FEBS letters 306(2,3): 189-192; Matsuura et al. 2001, J Biol Chem 276(48):45261-9), the truncated hrtrRNASE T2-50 (SEQ ID NO: 2) and hrtrRNASE T2-70 (SEQ ID NO: 3) contained 6 and 5 cysteine residues, respectively (FIG. 1).

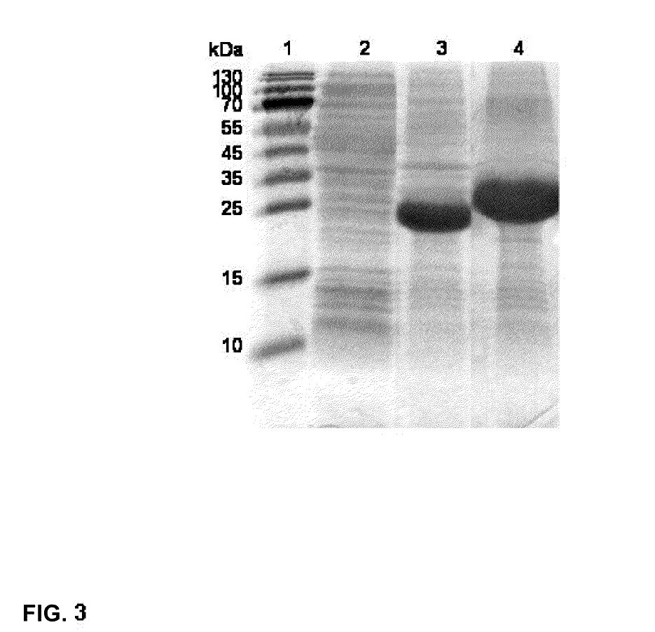

SDS-PAGE analysis showed that the hrtrRNASE T2-50 (SEQ ID NO: 14) and hrtrRNASE T2-70 (SEQ ID NO: 15) from the bacterial lysate, migrated at 19 kDa and 21 kDa, respectively (FIG. 3), corresponding to the migration of full-length human RNASET2, less the truncated portions. Lysate from bacteria containing the insert-less vector (Mock) contained neither of the truncated forms. Lysate from bacteria transformed with full length human RNASET2 contained either no human RNASET2 or no significant amount of protein corresponding to human RNASET2 (results not shown).

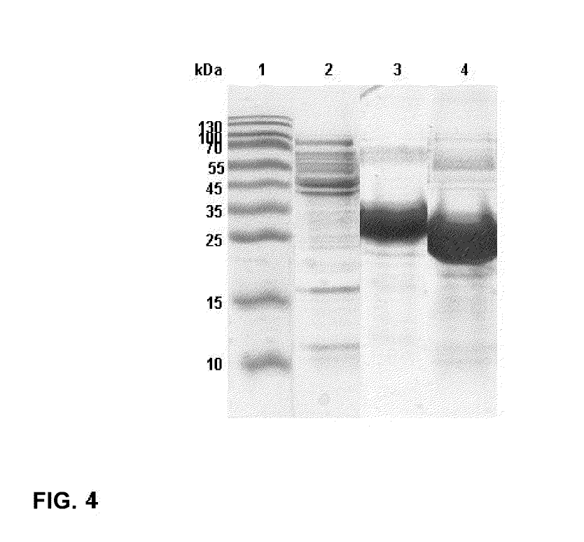

The bacterial lysates (300 ml lysate from 1200 ml of the original culture from each truncated protein or Mock) were loaded onto a HisTrap™ affinity columns and eluted with an imidazol gradient (5 to 500 mM). SDS-PAGE of the eluted fractions showed that a high degree of purification (>98%) of both hrtrRNASE T2-50 (SEQ ID NO: 14) and hrtrRNASE T2-70 (SEQ ID NO: 15) (FIG. 4) was obtained. Yield of purified hrtrRNASE, measured after lyophilization of the purified recombinant proteins was in the range of 100-200 mg protein per liter bacterial culture, and typically 120 mg per liter culture. Thus, constructs encoding truncated forms of human RNASET2 can be accurately and efficiently transformed into bacteria and expressed, resulting in a high yield of the recombinant RNASET2 protein.

### Example II

### Human Truncated RNASET2 Binds Actin Efficiently

Actin-binding activity of the purified human recombinant truncated RNASET2-50 was examined as follows.

Actin Binding Assay:

A. Solution actin binding assay: The solution actin binding assay was as previously described (PCT WO 2006/035439, Smirnoff et al. 2006. Cancer, 107(12), 2760-2769). Actin (10 μg) was mixed with 10 μg purified hrtrRNASET2-50 and 20 μL Buffer G (2 mM Tris pH 8.0, 0.2 mM CaCl, 0.2 mM ATP). The mixture was incubated for 30 min at room temperature; then, the cross-linking agent 1-[3-(dimethylamino)-propyl]-3-ethyl-carboimide methiodide (EDC) was added to a final concentration of 50 mM and incubated for another 30 minutes. The reaction was quenched with an equal volume of sample buffer and the cross-linked complex was separated on SDS-PAGE, as described above. From each reaction mixture, 18 μl (9 μg of each protein) samples were separated and stained with Coomassie Blue to visualize the proteins, and two samples of 1 μl (250 ng of each protein) were exposed to rabbit anti-hrtrRNASET2-50 (polyclonal rabbit-anti-hrtrRNASET2-50 was prepared at Anilab-Rehovot, Israel) or rabbit anti-actin (Sigma-Aldrich Company, St. Louis, MO.; Cat A2066) for immunodetection of RNASET2 or actin, respectively The membrane after blotting was blocked overnight at 4° C. with 5% (weight/volume) skim milk in TBS that contained 0.25% Tween 20 (TBST) and was washed twice for 10 minutes each with TBST. Then, the membrane was probed for 1½ hour against polyclonal rabbit anti-hrtrRNASET2-50 (1:1000 dilution in TBS) or rabbit anti-actin-IgG (12 μg/ml in TBS) respectively, and reacted against 5 μg/ml alkaline phosphatase goat anti rabbit-IgG (Chemicon Int., Temecula, Calif.; Cat AP132A) in TBS for 1 hour at room temperature. Signals were detected by incubation for 5 min at dark conditions, with 10 ml Develop Buffer for Alkaline Phosphatase, pH 9.5, containing 1.7 mg BCIP (5-Bromo-4 Chloro-3-indolyl phosphate p-toluidine salt, Sigma-Aldrich Company, St. Louis, Mo.; Cat B8503) and 4.1 mg NBT (Nitro Blue Tetrazolium salt, Sigma-Aldrich Company, St. Louis, Mo.; Cat N6876) as substrates.

B. Solid phase actin binding assay: The solid phase actin binding assay is modified from Mejean et al. (1987; 1992). Briefly, each well of a 96-microtiter plate was coated with G-actin (500 ng/100 μvl/well) in 50 mM buffer carbonate pH 9.5 for 1 hour and then washed with TBS. The coated wells were blocked with 3% BSA in 200 μl TBS for 1 hour and then washed with TBS. hrtrRNASET2-binding was performed using 1:2 serial dilutions, starting from 125 ng/well of the protein. Binding was carried out in 100 μl TBS for 1 hour. The wells were washed three times with TBST and incubated with rabbit anti-hrtrRNASET2-50 primary antibody (1:500 in 100 μl/well TBS for 1 hour followed by three washes with TBST) and then with conjugated goat anti rabbit-HRP second antibody (Pierce, USA; 1:10,000 in 100 μl/well TBS for 1 hour followed by three washes with TBST). Actin binding was revealed by detection of absorbance at 655 nm using a 1-Step™ Ultra TMB-ELISA (Thermo Scientific, USA).

Results

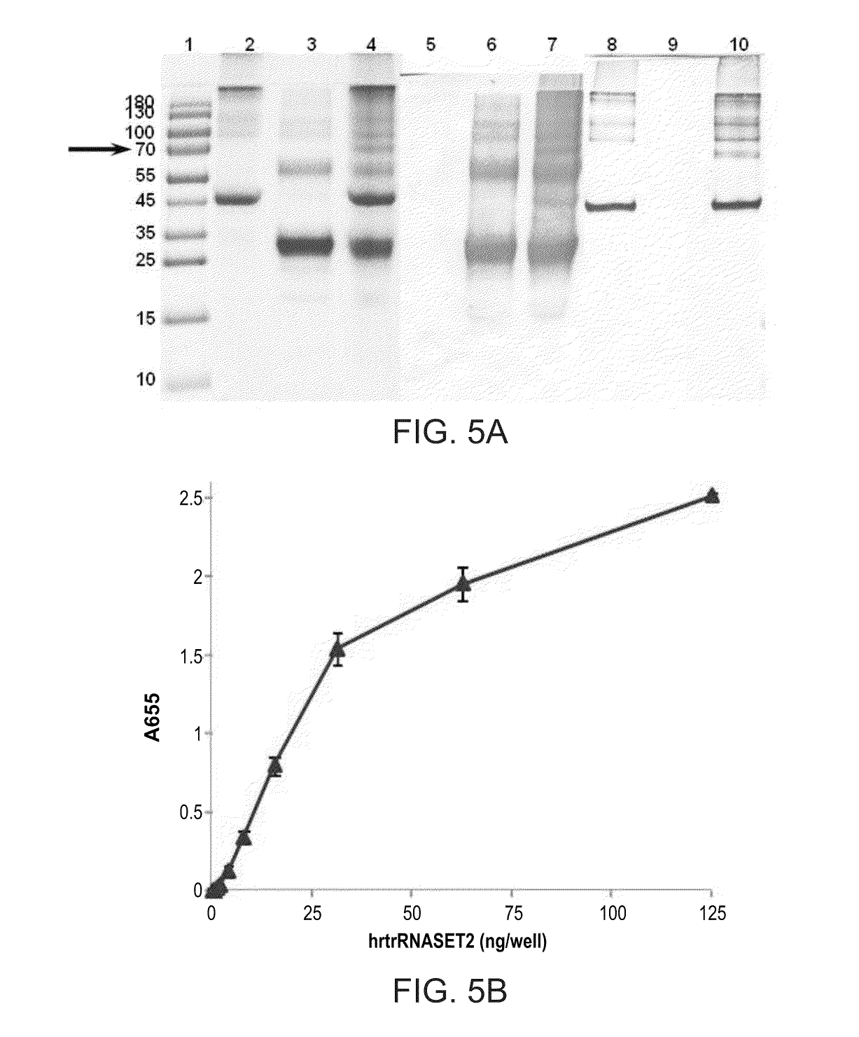

Actin binding of hrtrRNASET2—As shown in FIG. 5A, hrtrRNASET2-50 demonstrated strong and specific binding to actin in the solution assay. Following cross-linking with EDC and separation of SDS-PAGE, Coomassie staining of the separated proteins revealed the high avidity of the truncated hrtrRNASET2-50 for actin (see FIG. 5, Lane 4). Immunodetection of the cross-linked reaction products with anti-actin and anti-hrtrRNASET2 also confirmed actin binding by hrtrRNASET2-50 (FIG. 5A, lanes 7 and 10). Both Coomassie Blue staining and immunostaining of the SDS-PAGE revealed a 63-kDa band representing a hrtrRNAST2-50-actin complex (FIG. 5A lanes 4,7,10).

Using the solid-phase ELISA assay, it was shown that truncated hrtrRNASET2 binds actin in a fairly linear, concentration dependent manner over a broad range of concentrations (20 ng/ml to 300 ng/ml) (FIG. 5B).

Thus, recombinant human truncated RNASET2 retains the specific and high affinity actin-binding capacity of the full-length human RNASET2.

### Example III

### Human Truncated RNASET2 Effectively Inhibits Cancer Cell Growth and Angiogenesis In-Vitro

The anti-angiogenic and anti-cancer therapeutic potential of full-length human recombinant RNASET2, similar to that of fungal T2 RNASE has been demonstrated (see PCT WO 2006/035439, incorporated in its entirety herewith). Anti-cancer and anti-angiogenic activity of the purified, truncated forms of hrtrRNASET2 from bacteria was examined as follows.

Colony-Formation Assay:

Human colon (HT-29) cancer cells were grown in 50-ml flasks (10⁵ cells per flask). The medium contained 7 ml DMEM supplemented with 10% fetal calf serum (FCS), 1% glutamine, and 1% antibiotic-antimycotic solution in the presence or absence of 1 μM hrtrRNASE T2-50 (SEQ ID NO: 14), hrtrRNASE T2-70 (SEQ ID NO: 15) or recombinant insert-less vector bacterial lysate (Mock). The cells were incubated at 37° C. in a humidified atmosphere containing 5% CO₂. After 48 h, 10³ cells/well were seeded in 96-well plates in 200 μl medium, in the presence or absence of 1 μM hrtrRNASE T2-50 (SEQ ID NO: 14), hrtrRNASE T2-70 (SEQ ID NO: 15) or recombinant insert-less vector bacterial lysate (Mock). After 5 days, the cells were fixed in 4% formaldehyde and stained with methylene blue. The number of colonies was counted.

Human Umbilical Vein Endothelial Cell (HUVEC) Angiogenesis Assay:

Freshly isolated HUVEC were maintained in M199 medium supplemented with 20% FCS, 1% glutamine, 1% antibiotic-antimycotic solution, 0.02% ECGF, and 50 units/100 ml heparin. They were then plated in a 96-well plate (14×10³ cells/well) previously coated with growth factor-depleted Matrigel™ (BD Biosciences), in M199 medium containing 5% FCS and 0.005% ECGF. The wells were supplemented with 200 μg/ml each of hrtrRNASE T2-50 (SEQ ID NO: 14), hrtrRNASE T2-70 (SEQ ID NO: 15), recombinant insert-less vector bacterial lysate (Mock) or PBS, with each angiogenic growth factor (1 μg/ml angiogenin, bFGF or VEGF) to a final volume of 120 μl. After overnight incubation at 37° C., the plates were photographed, and the extent of tube formation (angiogenesis) was assessed.

Results

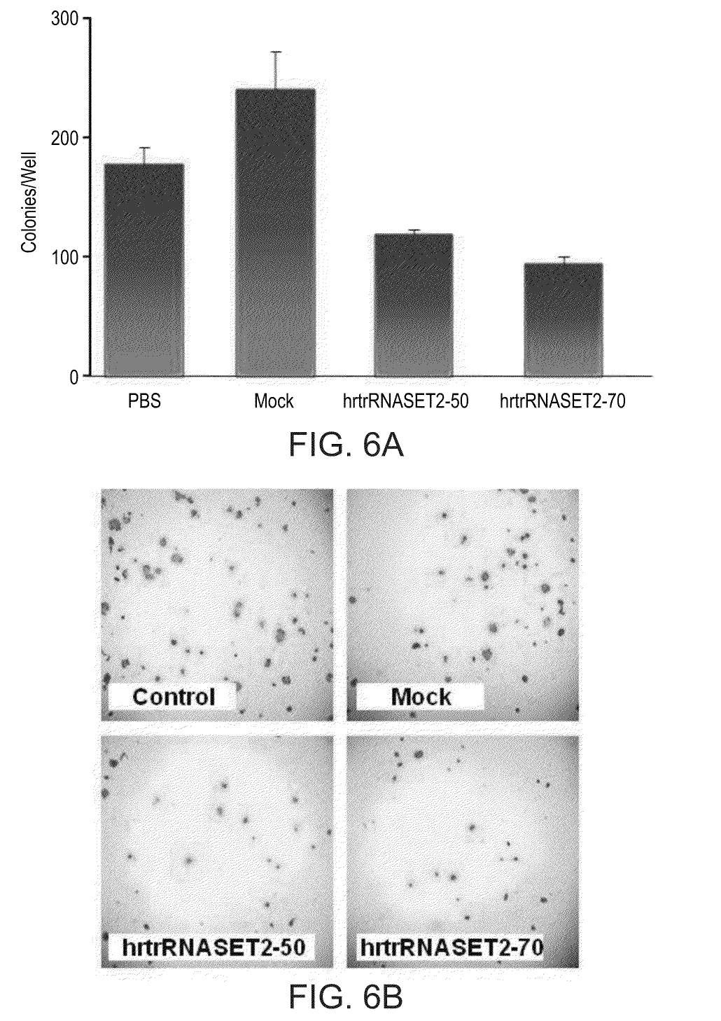

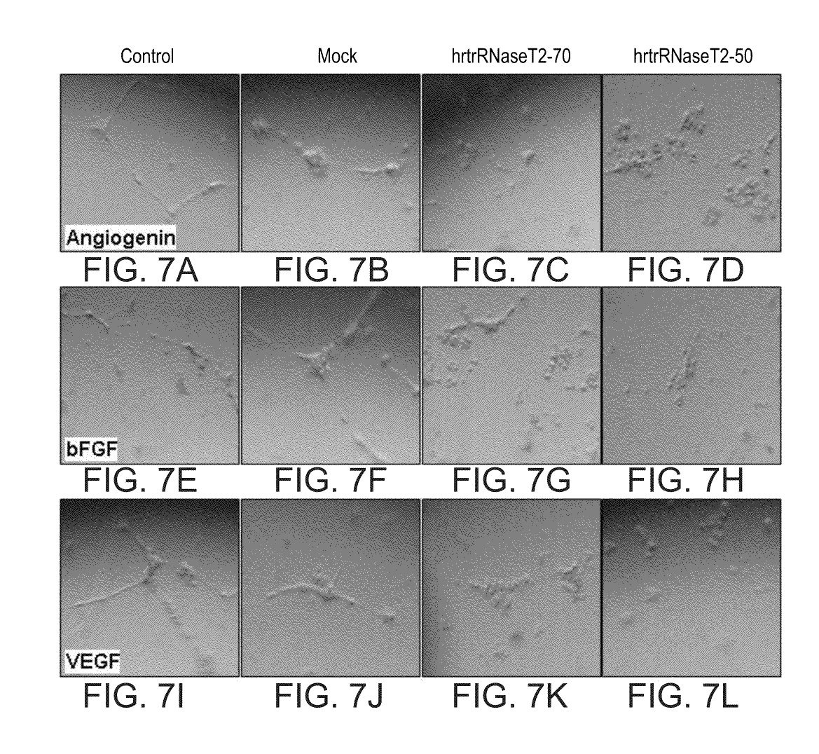

Both truncated forms (RNASET2-50 and RNASET2-70) of RNASET2 had a significant inhibitory effect on colon cancer cell colony formation compared to control (PBS), while insert less vector bacterial lysate (Mock) actually induced cancer cell colony formation (FIGS. 6A-6B). When assessed in the HUVEC Matrigel™ assay, both truncated forms of RNASET2 inhibited angiogenin, bFGF and VEGF-induced HUVEC tube formation (FIGS. 7A-7L). No effect was observed in control or Mock.

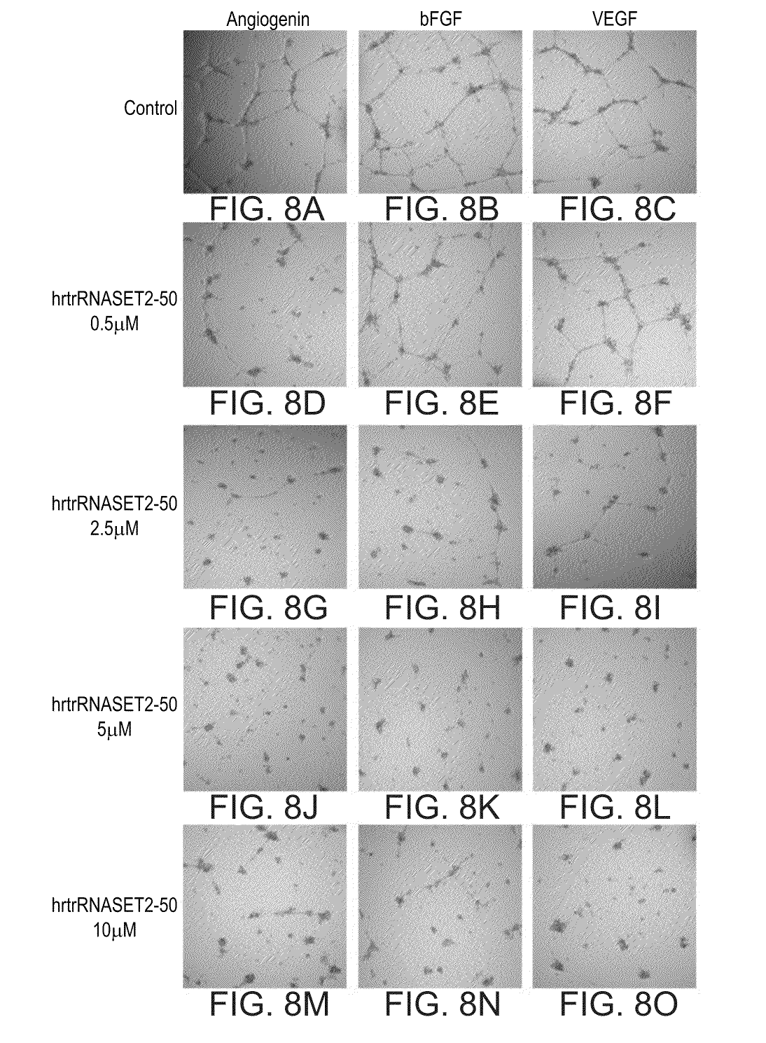

In order to further examine the effect of different doses of truncated RNASET2 on angiogenesis, an additional HUVEC tube formation assay was done, using the same protocol, with hrtrRNASET2-50 at concentration of 0.5, 2.5, 5 and 10 μM. PBS was used as control (FIGS. 8A-8O).

hrtrRNASET2-50 inhibited HUVEC tube formation at a dose-responsive manner in the presence of each of the growth factors examined (FIGS. 8A-8O). Note that tube formation was significantly inhibited at hrtrRNASET2-50 concentrations of 0.5 μM, and completely prevented by hrtrRNASET2 concentrations of 5 μM and greater. Angiogenin-induced tube formation was inhibited at 0.5 μM hrtrRNASET2-50, while bFGF and VEGF-induced tube formation was inhibited at 2.5 μM hrtrRNASET2-50.

Thus, these results clear demonstrate the anti-cancer and anti-angiogenic properties of the truncated hrRNASET2.

### Example IV

### Human Truncated RNASET2 Effectively Inhibits Cancerous Tumor Growth and Angiogenesis In-Vivo

The anticancer and antiangiogenic effects of hrtrRNASE T2-50 (SEQ ID NO: 14) and hrtrRNASE T2-70 (SEQ ID NO: 15) were demonstrated in in-vivo therapeutic models. The study was performed in athymic (balb c, nu/nu mice) 6-7 weeks old female mice in the HT-29-derived xenograft tumor development assay.

Xenograft Model:

hrtrRNASE T2-50 (SEQ ID NO: 14): HT-29 cancer cells (0.5×10⁶/mouse) were injected subcutaneously into the left hip of the athymic mice. When the tumors were palpable (10-13 days after HT29 cell injection), 5 mg/kg each of hrtrRNASE T2-50 (SEQ ID NO: 14) or insert-less vector bacterial lysate (Mock) in PBS, or PBS alone were injected into the tail vein, every other day (three times a week), totaling 9 injections altogether. During the experiment, the tumors were measured twice a week. After 21 days, mice were sacrificed and the tumors or the area of injection were excised for size measurements and histopathological examination. Five mice were used for each treatment. Tumor volume was calculated using the equation (length×width)/2.

hrtrRNASE T2-70: A similar HT-29-derived xenograft model was performed with hrtrRNASE T2-70 (SEQ ID NO: 15) (5 mg/kg in PBS) administered IV to mice with tumors induced by subcutaneous injection of HT-29 cancer cells (1×10⁶/cells per mouse) into the left hip. Eight to ten mice were used for each treatment.

Blood Vessel Analysis:

The effect of hrtrRNASE T2-50 (SEQ ID NO: 14) on angiogenesis was determined in the median tumor cross-sections. In each cross section, angiogenesis was scored following Matsuzaki et al (2007, Calcif Tissue Int. 80: 391-399). Using image analysis software (Image J; NIH, Bethesda, Md.), a binary image was created using a threshold value midway between background (white) and blood vessels (black). The number and size of all black objects (blood vessels) greater than 10 pixels in size were determined using the particle analysis function of Image J. Vessel number, total vessel area and the relative area (the ratio between total blood-vessel area and tumor-section area) were determined from these data. From each tumor section, 2-4 different field areas were determined. Means were compared by using an analysis of variance. Differences were considered statistically significantly at P<0.0005.

Results

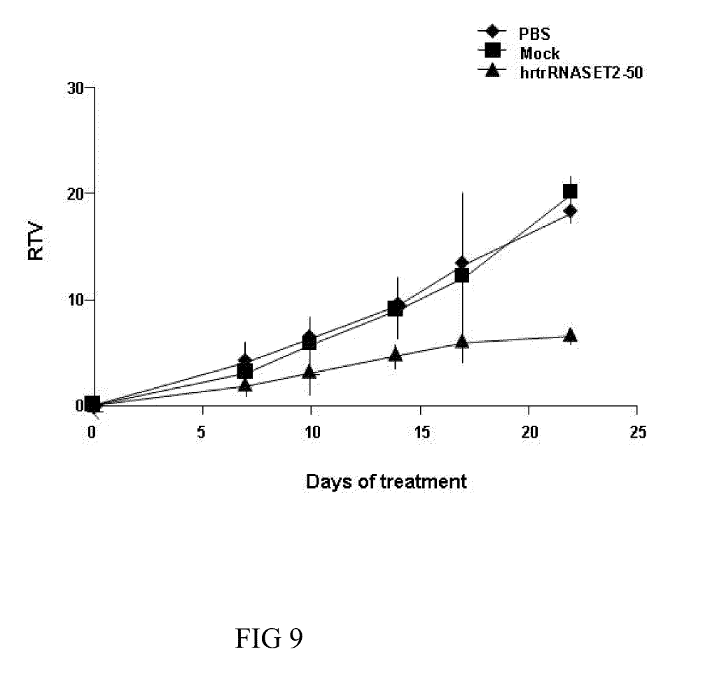

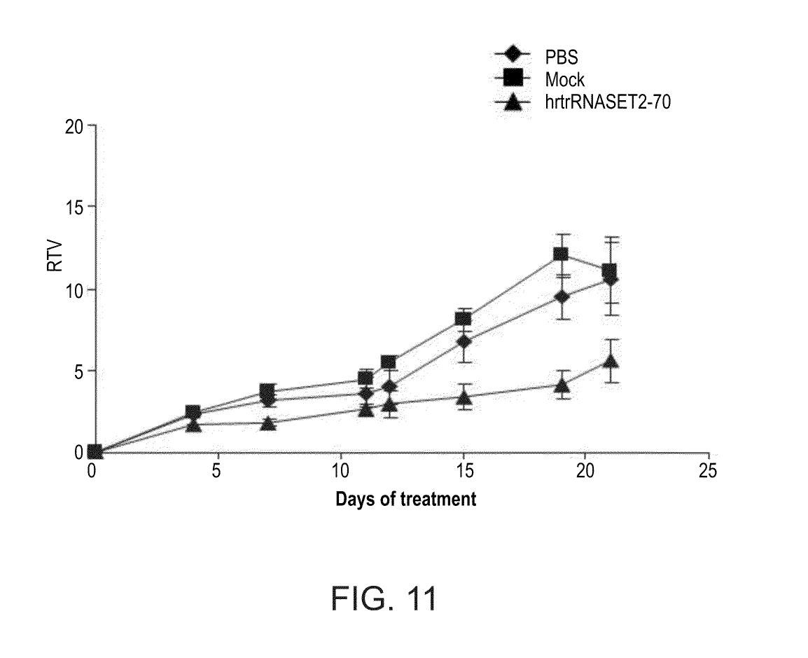

In hrtrRNASE T2-50 (SEQ ID NO: 14)-treated mice, a significant reduction (60% to 67%) in tumor weight was observed compare to Mock-treated mice and controls (FIG. 9). In hrtrRNASE T2-70 (SEQ ID NO: 15)-treated mice, a significant reduction (50%) in tumor weight was observed compare to control and Mock-injected mice (FIG. 11).

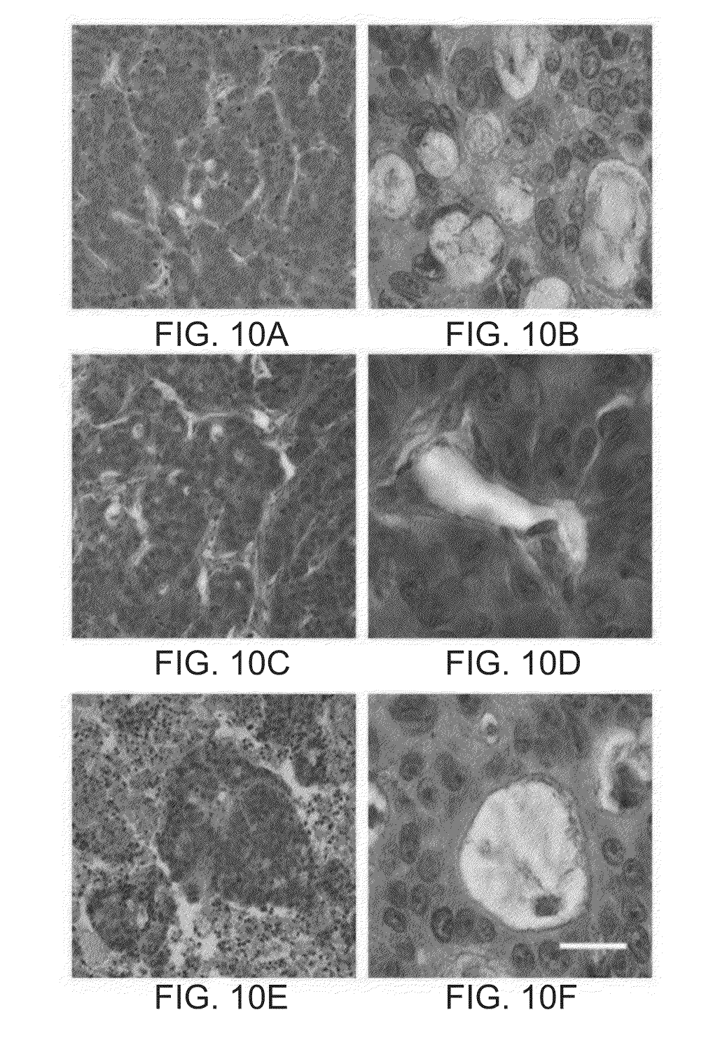

Histologic analysis of the tumors and surrounding tissue demonstrated the inhibitory effect of hrtrRNASET2 on the tumor cells and tumor angiogenesis (FIGS. 10A-10F). In control and Mock-treated mice, a wide invasive area with abundant HT-29 cancer cells was observed, accompanied evidence of enhanced angiogenesis (note the numerous blood vessels) (FIGS. 10A and 10C, respectively). The endothelial cells were intact, and tumor cells were evident, attached to the blood vessels (FIGS. 10B and 10D, respectively). In mice treated IV with hrtrRNASE T2-50 (SEQ ID NO: 2) (FIG. 10E), the tumor cells were concentrated in clusters surrounded by abundant necrotic tissue. High-power magnification (FIG. 10F) shows that when the tumor cells detach from the blood vessel, a substantial loss of endothelial structure is observed (FIG. 10F).

Analysis of the tumor blood vessel parameters indicated a significant inhibitory effect of hrtrRNASE T2-50 (SEQ ID NO: 14) on tumor blood vessel number, total vessel area, and relative area compared to control and Mock-treated mice (P<0.0005, Table 1, below). The number of blood vessels was similar in the tumors from Mock-treated and control mice. However, in keeping with the cancer cell colony-promoting effect observed in-vitro (see FIG. 6A), angiogenesis (total vessel area) was significantly greater in the Mock-treated mice than in the tumors from control (PBS).

TABLE 1
Control values of tumor blood vessel parameters at each treatment
(Mean ± SE)

| | Control | | hrtrRNASET2-50 |
|---|---|---|---|
| | (n = 10) | Mock (n = 10) | (n = 16) |
| Vessel number | 466^{a} ± 42.1 | 427.5^{a} ± 32.1 | 163.3^{b} ± 29.1 |
| Total vessel area | 8,527^{a} ± 966 | 13,621^{b} ± 1,444 | 2,915^{c} ± 505 |
| (μm²) | | | |
| Relative Area (%) | 5.6^{b} ± 0.7 | 9.0^{a} ± 1.0 | 1.9^{c} ± 0.3 |

^{a-c}Results with different letters are significantly different, P < 0.0005

Thus, the results of both the in-vitro and in-vivo assessments of the biological activity of truncated hrRNASET2 shows that the presence of truncated hrRNASET2 is sufficient to effectively inhibit tumor angiogenesis and colon carcinoma tumor growth. Further, the in-vivo assays demonstrated that therapeutic amounts of truncated hrRNASET2, administered intravenously, could reach and effectively inhibit growth and angiogenesis in cancerous tumors. Thus, the anti-cancer and anti-angiogenic biological activity of truncated hrRNASET2 is similar, if not identical to that of the full length RNASET2.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An isolated human truncated RNASET2 devoid of ribonucleolytic activity and having an anti-angiogenic activity.

2. The human truncated T2 RNase of claim 1, devoid of the amino acid sequence corresponding to amino acid residues 1-32 of the N-terminus of SEQ ID NO: 1.

3. The human truncated RNASET2 of claim 2, comprising an amino acid sequence at least 95% identical to SEQ ID NO: 2.

4. The human truncated RNASET2 of claim 2, comprising an amino acid sequence as set forth in SEQ ID NO: 2 or 14.

5. The human truncated RNASET2 of claim 1, devoid of the amino acid sequence corresponding to amino acid residues 1-49 of the N-terminus of SEQ ID NO: 1.

6. The human truncated RNASET2 of claim 1, devoid of the amino acid sequence corresponding to amino acid residues 1-52 of the N-terminus of SEQ ID NO: 1.

7. The human truncated RNASET2 of claim 6, comprising an amino acid sequence at least 95% identical to SEQ ID NO: 3 or 15.

8. The human truncated RNASET2 of claim 6, comprising an amino acid sequence as set forth in SEQ ID NO: 3 or 15.

9. The human truncated RNASET2 of claim 1, devoid of the amino acid sequence corresponding to amino acid residues 1-69 of the N-terminus of SEQ ID NO: 1.

10. The human truncated RNASET2 of claim 1, devoid of a cysteine residue at least one of amino acid coordinates corresponding to amino acid residue 25, 32 or 52 of the N-terminus of SEQ ID NO: 1.

11. The human truncated RNASET2 of claim 10, devoid of cysteine residues at amino acid coordinates corresponding to amino acid residues 25 and 32 of the N-terminus of SEQ ID NO: 1.

12. The human truncated RNASET2 of claim 10, devoid of cysteine residues at amino acid coordinates corresponding to amino acid residues 25, 32 and 52 of the N-terminus of SEQ ID NO: 1.

13. The human recombinant truncated RNASET2 of claim 1, further comprising a recognition entity peptide sequence.

14. (canceled)

15. The human truncated RNASET2 of claim 1, having actin binding activity.

16. (canceled)

17. A pharmaceutical composition comprising the human truncated RNASET2 of claim 1 and a pharmaceutically acceptable carrier.

18. An isolated polynucleotide encoding the truncated human RNASET2 of claim 1.

19. The isolated polynucleotide of claim 18, comprising the nucleic acid sequence as set forth in SEQ ID NOs: 4 or 5.

20. An expressible nucleic acid construct comprising the isolated polynucleotide of claim 18.

21. The nucleic acid construct of claim 20, wherein expression thereof in bacteria produces at least 50 mg human truncated RNASET2 per liter bacterial culture.

22. A cell transformed with the nucleic acid construct of claim 20.

23. The cell of claim 22, wherein said cell is an E. coli bacterial cell.

24. A bacterial culture comprising a plurality of cells of claim 23 and expressing at least 50 mg truncated human RNASET2 per liter culture.

25. A method of inhibiting angiogenesis in a subject in need thereof, the method comprising administering a therapeutic amount of the truncated human RNASET2 of claim 1, thereby inhibiting angiogenesis in a subject in need thereof.

26. (canceled)

27. The method of claim 25, wherein said inhibiting angiogenesis is inhibiting angiogenesis of a tumor.

28. The method of claim 27, wherein said tumor is a benign or malignant tumor.

29. The method of claim 27, wherein said tumor is a primary tumor.

30. The method of claim 27, wherein said tumor is a metastatic tumor.

31. An antibody recognizing a human truncated RNASET2 polypeptide having an amino acid sequence as set forth in any one of SEQ ID NOs: 2, 3, 14 or 15.
